SEQUENCE LISTING (113 sequences)

SEQ ID NO: 1 - DNA, 301 bp, Glycine max
  misc_feature (18)..(18): n is a, c, g, or t
ccaaccagtctttccttnttgttgttttccttcatatgtcaacttggtgtaactcaatgttgagtgttgacgtagcattgcataataataataatggcagcatagagttaaattcaatttattgctcctagattgtaatgtagcttctataactagtgcctctaatcttttgcttattacttccccaaccccaacaacaanggatgcaaaatatcatcaggctctaacacagatantagataattgagtttttttaacatgtcaaatcttgattatgtgtataaaaaagattttattgaga

SEQ ID NO: 2 - DNA, 301 bp, Glycine max
  misc_feature (20)..(20): n is a, c, g, or t
attaatcttatattattatnaatttaattgtctatcattaatattataaaaaataaaataaaaaataattaatatcatattaaaaaattaaaataataattattttcttttagatgataattataaatataacgaggcagcatttgattaaaattacttaatttttaaatatttttatctcaaaatgttactttatggttnaaacttaaaaagagtgaaaaaggtggaaggaaacgacgtcgtggagtacgttgcaaatggataacatcactcacagaatcaaaagacacagaagagaggt

SEQ ID NO: 3 - DNA, 301 bp, Glycine max
  misc_feature (80)..(80): n is a, c, g, or t
taagagaacttgacactatattttaacccaaaattttaagatttaaatttatgaatcttctcctcacttattgaataaanttagtntttggcacgtaacaaacaaatccaatttttttttagggaacgaattttcatttaggaaaatggactaaatatatcataacttactataaactatttaaatgtatagagatgtacngtcaacaagttgtaagcagtgttaacaacactactcagaatcagttcgagtctggagttataattagtcagtttattttgcagttttttaccaaattatc

SEQ ID NO: 4 - DNA, 301 bp, Glycine max
  misc_feature (201)..(201): n is a, c, g, or t
gattgttaagaacatatttacttcggatgttttcatcccacgtatagcttgttgtctttgtttaatgaagttagatcatcaattgcgtttgagtgtcttagaaaaacacacattacttctactcatctcatactaatttggaaatgttgtgaaggatgcattactatctatatttttaaaaattatttattactattactntggtgtctcatttgttgagtttcacagcacagaaccaagaaccaaacattttattaagcatatttcaagtttcagangtaaggaataatagacgtctgga

SEQ ID NO: 5 - DNA, 301 bp, Glycine max
  misc_feature (3)..(3): n is a, c, g, or t
ttnccgatagatattcattctgtataattcttttttgggcattaggctctttaggcccaataattacattattatctttgaattagatcagtgtagttcccttagtttgctgaattgtaagatacttcatttgtacaaaagccatttcttctaaaatgcataaccttggcaatttcgttatcaatttggcgacccgtgatnatttttctagctatcgcctaggtgtttctagattctgctggctgagctttagtattgggtcatggtttgtttacggcaggngttttaataaaatatgttc

SEQ ID NO: 6 - DNA, 301 bp, Glycine max
  misc_feature (55)..(55): n is a, c, g, or t
caaagacaaactatattccccccattcagaaaaatagacactttctctagctacngagggataatcacagttagagccatgctttaaatcacaatgatcaccgttgcagttaccatgcttttccntaattggtgattttagaagccactcctctaatgtaagctccttcccctttttctgatgctgctcctctttattctncttcttttgcatncccttcttcctaagaaagtaagtttgcttgatatttttggcaatccatactactgaactggcctttacaaaacctgagtcctcctta

SEQ ID NO: 7 - DNA, 802 bp, Glycine max
  misc_feature (186)..(186): n is a, c, g, or t
tttcttttgcagatgcagacacagaaacagtttctaagcaatgttccccaacagaaatttcaaggatgaaaaagttaagtaataaggtagattcaagttaaggtgtggaatcattctatggatctgtttgtttaaatgtttcaataagatgatcaagcgttgaaggttcactgtgactgatatcgnagttcgaaggataagatggtttttgggtttcttgtcaagtctctgtcaatgtcccagtgacgacaaaatcacggtaggttaaaagttaaaacagatcagaataacatttcttggaatagccataagaagattcaactgttggatcttttcagatgataacccaaataccctaacttaatccacactccagctccttgcttgaaaggaaacgggagtttaagtatgatatggttaaacctaccaaacttattccaatttagttttcatgacaatttctgtgaatataatgtggcccggctagacagctgccaaaactcatcactatcttctaagaaaatcattaaaaatgtaatagatttttatgccaaaccaaattgtactaatctctgaaatggaacttaagcaacaagtaattatgatgtactaaggaatttcacagctgagattcaaatcctcagggtcaatgggttaatgaacgcgaacctgaatttgagtgcaagacccaaacanaaagaaaaagagaattgtcccagacagacaaactganatntatctagcagacatctctttatatgtatcacatttcctttgacaacaagccaatctcaataactaa

SEQ ID NO: 8 - DNA, 301 bp, Glycine max
  misc_feature (201)..(201): n is a, c, g, or t
tccaaatgagtaatgacagcatcactgtgaactacctatatttctgtttagtgtataatatataacccttttgcaacgtgacaacattttcatttcatcactttccttggaccataacttgcattctcattctacgtaagccatccattataataataattataattttatcatcaattgatcatgcgcgggatagcctcnatatccctacatataaatataattgctgcatttggggtggcctacgtttcagccattttaatttattgagtaatatattatgtatctatcattgcatgta

SEQ ID NO: 9 - DNA, 301 bp, Glycine max
  misc_feature (57)..(57): n is a, c, g, or t
gtctcacgggaattagttcataattaaagatttacttgataaagtactccttctatncctcatttatgtttagttttaatttagttttataaagtatttttgttaaattaaattaatatttccgacaatttaccanattaacattattaatcatgattgatgtggcaattgtcgatcccgtgttaaatagacgaaaagacnaatttaatctaataaatacactttaagaagctaaagtaaaacttttaaaataagatcaaactaaaacttaaaaaaaaaagacganagtcttaaaaaggat

SEQ ID NO: 10 - DNA, 301 bp, Glycine max
  misc_feature (145)..(145): n is a, c, g, or t
atgtcacacataggatattggttggatgacttaaatggagaaaagtttcagaggtcgtttgggaacgtaaagtacataccaaacttaaaggcaagttttgtgtatagttagacatctaactatactatatgatagtgaatgttgngttttaaaggaacatgaaagaaaaataaaagtagcaaaaatgagaatattaagatngatgtgtggtcatataagaaaaagaagaataccgaatgacgttatacgataaaagattggtttagtatccattgggaaaaaaaatgagaaaatcagttaa

SEQ ID NO: 11 - DNA, 543 bp, Glycine max
  misc_feature (41)..(41): n is a, c, g, or t
gtggttagagggagggtgaggatgcagggaattaaattagngtttacaataatgttggtggttgctggcattgtagcagtgccacgcatctcgggtcaagtatgcaatggtaatttgggtactattcaagaggaatgcgaggagtactataaacccggagggccaactattccgccatctacagaatgctgcaacgctctgagaaatgttgatgtgccctgcatgtgccgacttgctaacaattttcagtcatacttcagcgggggaaaagttgtctataccctcaggcagtgtggaaaagatgttcctcccggaaccacctgcggaggtctctcttttcttcaaaaatgctagtgtgattagggtgttagttaaggaattcaagaaaaatatttttcatctttttatcttctcctatatatatatatatgacttctataataaatactttttatttcaggttaccgtgctccccctgcttctactcaggtgtgaacgtcagcattgaagacgtatgcatacaacttcccttaccctcgggat

SEQ ID NO: 12 - DNA, 301 bp, Glycine max
  misc_feature (46)..(46): n is a, c, g, or t
ggatcacacactcatttatagaagaaacaaaaggcagttattaacntgtgattaattcaatatttgactttctcttaacccaaagctgaatggatacgtttcctcacctttttaatttgtcacctaatttcacacaggaaaaaagctaattaatgtgatatttaaaatctaatgggaaaaagattatcatacatttatcgnatttttacacccgattttcatctaatgcgcctatcttgtttgtgtgggtgtgtgaatttgtaaaatgatcattacattataaaaagaaaaacaatatcgt

SEQ ID NO: 13 - DNA, 301 bp, Glycine max
  misc_feature (4)..(4): n is a, c, g, or t
ctanactttccatcacattacttgttacttctatccctttgtgcctctgcttttgtttattcttcctatctctcttgttccacaccaaaatggggtgaatgtttgcaaatttccaaaaacaaaattgaagcagctgagaggaagggcactaggcagaagtaacatcaatcttaagaccaagagacactatttaataaacgngaataattagcaaaattggacccacactatgactaaaaagaacacatttccgaaagcatagataactaaatacataaatgcacgtgggtcttatagtaaa

SEQ ID NO: 14 - DNA, 301 bp, Glycine max
  misc_feature (126)..(126): n is a, c, g, or t
ctgtggattctcttatgcactccttttgacaaagagccagcaagtgcattgctctttattgtgtttgaaactgccctcagttgtacgtgtgttttttatgttgtgaaactgaaattattcaatgtnagtgtttttgcctatgataaataagctaatgtgagtatccttttgactgangaggaaaactttctttgacctgtnctgaaccgtgagaaaccaaggagtgacagtcgaaaatctatttgcccttattggagttgacgtttgaaggggcagtgctggacgaaattatcatagggca

SEQ ID NO: 15 - DNA, 301 bp, Glycine max
  misc_feature (37)..(37): n is a, c, g, or t
ataaacgaaaaggggttaaatgagagattaaatactnttttaaaaaaattaaacaaaaacttgtcaacaacgtcnatgtttattaccagagttaaaaaaaaaanaatctattttttactttaaaaacgggagacacgttcgttagggcagggtttagtctattatggcatgtgcatccccntnttgaaattaggcagcttntgagtttctgatatttggggagaccagactcccctagtgttatttgtgtgtagttttactttgggtttgggccctttgccttccaaaaaataaaaaaggt

SEQ ID NO: 16 - DNA, 301 bp, Glycine max
  misc_feature (201)..(201): n is a, c, g, or t
atgagtggtcaatatttttttaaacggtcaactcaagcgacttaattgaaagatcaaaattgtgaaaaattggaacttgaaaaattaaatgttacaatttgaaacttgggatcaaaatcaccaaatgttaaatttgagaattaaaattacaattttaaaaaacatacacaccacatggatgatgttgactttcatgcttgnattcgtattcccttcaaactcacttgagctgatgcaagaataatgtaggattagaatgaacccttaattataagaattcggatcaaacaatgccatttaa

SEQ ID NO: 17 - DNA, 301 bp, Glycine max
  misc_feature (201)..(201): n is a, c, g, or t
tcaacattagttcacaaaaatggagtttctactttataaattcatacaagaatgaaaatatttacaatcttaacaaaaaaaaccgtaaattagagttactttgcaataaaagctcatgaataccaattaacaccgttgcattttttgtgatggtcattgtccatctgacctttgttatcaaacatcttctcttttgttctnttctttattctcaaacttcttttcttatcttctcggttatgaaatttgtcttcttttctttattctaatttttttttgttctctatttataaaaatacaa

SEQ ID NO: 18 - DNA, 301 bp, Glycine max
  misc_feature (201)..(201): n is a, c, g, or t
gttttgtttattttgaaggcagacaacggctacaaaacagcgttgcgaccgtgcaagaaggaacaatgtgtgtaggctataagaggagacgacctggtttaaaagtaaacctttgttaggaattgccactcactcccgattttactatttacaccccacgtgctttaaaaatgacattattccaaaagcacccttttcatngaaacattattctgaaaaattacacaaccagatcatgtctccattgcgtaagagggagcgcaacaaaacaaaaataaaattatcaaggaaatcatgcttt

SEQ ID NO: 19 - DNA, 301 bp, Glycine max
  misc_feature (201)..(201): n is a, c, g, or t
cataccaacttaatcataaaatttatgcgttatcgcaatttgcaaaaaaagaatcaaattttgcacgtttattattaatttttacctaaccgtttcttttaattttagtagactactctaaatattattcttttagttattacgaaaaaattcaggatgtttccgttggagcaatggactatgatattaccaaagaagttnaagcaatgcaattattgtcagccccatatagcttttacaagttagccatcaacatggctcgaattttngctctaagctcataagaaacatcagttatttc

SEQ ID NO: 20 - DNA, 301 bp, Glycine max
  misc_feature (201)..(201): n is a, c, g, or t
agataatcatataataaaatagtctcacctctccacctctatgtccctgcaggttttttccaaaggaaaagaaaggtcttcgcaggtaaaatgtgactaatcaactagggaagatcaatgcataagcagacaaggacattactgagctttgcttataagacagaaagcaggggaagtttgagagaaaagtaaagggaaggnaaattagactcactaactggctcagaataattatactacattagcttaaccctagacctttacttatactggagctatgaccaccaaatgacatttatat

SEQ ID NO: 21 - DNA, 301 bp, Glycine max
  misc_feature (201)..(201): n is a, c, g, or t
aaatctttcaaacttttctgaaaatgtctatattgtagcaacttttcaagcaatattgacagaaaaaatgcttatcagttgtcacatcacattaaacatggcatcagaaagtctcgaaccaacacaaacagaagagaagaaaaataacttcaggggaaaaaacatgtacagaataaattaaatacagggcataggataagnacttcacatgcgtggaagaagctcttgcacagcaccaattttgttgaaccagttatagcaagtttctttggcatcagcacaaatatcccttgcttgaaag

SEQ ID NO: 22 - DNA, 301 bp, Glycine max
  misc_feature (201)..(201): n is a, c, g, or t
gaacccgggaaaaaggcctgagatgattggaaccaaagccatcatagccattaaacatggcgacggaacgtggaaaatagacacatacaacgtcactaaggagaccagaaacggttgcagccttcttccgtcgaaaatcgcgtttgtgaccaacatgtcagttgagcaaaaggtcgctaataggaacaccatgtatgctanattggtccttccttctgaagtgtacaacgttacgaagctgaaccatgtgtggcaagttgggtatgacattgaagatggacatccccttggacatccaacc

SEQ ID NO: 23 - DNA, 301 bp, Glycine max
  misc_feature (35)..(35): n is a, c, g, or t
tgaattactaatagtgtaaaaatatccccagactntgctgagtgttgtatttgtcaacctctttattaagctgcatattaacatagatctctacattttctgctaatttataattagttactaacttcgttcaagtgagatattggaatttagtgctatactaatactgccgcagtgcctggacaaatttggctgtctatnctttgttcctttgaaaaaaaagatgatgtgctaatagacatattcttatttatctaaaaagccgcatctaagttaaccaactaaccattaattttagaag

SEQ ID NO: 24 - DNA, 301 bp, Glycine max
  misc_feature (14)..(14): n is a, c, g, or t
agttccaaaaaaanaagcattgattaaaaaaaaacccttggcaatatttcatagaaaaatattgacgcaaaaagagaattgtggtggggacgtggggttggaattagtaccaagaacaagaccaaaaaacatgctattgctagtaaaaggcgagctgtaattaagaaattaatcaaataaagaattgtttcccagcataantgggattggttattcattgagtagttgcaaacacttcgatcggtggaatgttatcttactagtacttttaatttttttaaacaaaaagaaagttttatta

SEQ ID NO: 25 - DNA, 301 bp, Glycine max
  misc_feature (9)..(9): n is a, c, g, or t
tgaagtacnaacttatgaattgtgggtcatagctatgcaagaagaatttgattgaaatttaagtctaaacattgntctcatcacntgaaaattaacttgttgctaggcaaaaccaatttatcatctacctatgatcgttccctataacaactttgacactgtagagtcacaaagtaaccttggatatccatagtggtctcngtgttgtctctttcatccctnttgttcttaggatttggtaacatttgtgccaataaatantaattgttncttgaatgtttttttgttgatcatagtgttc

SEQ ID NO: 26 - DNA, 301 bp, Glycine max
  misc_feature (201)..(201): n is a, c, g, or t
tgattataatcatgttcatgtgatgttatgtgttacctgtagttgcacttttagtgagaaagccatggaatccacatactgttgtggggtgcttttcttacgaagatccgatgattcaaagtatccaaatatatcattgctgccaatcaccacaacgaatattgattttgagagatgcttctgtagaccagctgctcctcntacttctcgagtcatttcctcatgcacaattgagtagtagtccatttgttttctcaacggtattgattgtctctgaaatgatatataataatatatattg

SEQ ID NO: 27 - DNA, 301 bp, Glycine max
  misc_feature (88)..(88): n is a, c, g, or t
aagaatgcaacacaacaacaacaacaacaaatgatggatcatatcgtaatgaagaagagggggcgtccctccaaggcagatctagctngtcgctctgacaaatctttggccacctcgcaaccagacctccgacgnagtcttcgtcgtcgtcggaatntgagttacaacattgactacctcaacaaggaagatgaggaccangaagaggatgagaagaggcacaacaagaagttaaagctcgtggtgaagttcaacaacaatgaagaaaaagacaaggatgaggatgactacatgccaagtc

SEQ ID NO: 28 - DNA, 301 bp, Glycine max
  misc_feature (201)..(201): n is a, c, g, or t
atagaatgtatgagtacacttttgttcgagaaattaaaaaaggaagtaattacattttagtttttggtattcaacaaaaattagagaagaacagacacaaatgagcaactaagaaaaaaaaatcaataggcaagaatgggtaggtattagaaatgctaggtatggtgttgtagaaaccactggcgtcgaactctaacttgnccacctttgggcgcaaaattcacaagcatgggcaccattgtagtgattgncgtggtggcatcatgtgtagctgaccacgatatataccaagtgtgacatt

SEQ ID NO: 29 - DNA, 301 bp, Glycine max
  misc_feature (13)..(13): n is a, c, g, or t
gaggagtttcctngtaggactttattttcanttgtgagggttagtctgtagccttcacttttgtgacaanctgggactgcaacaatgttgcttcaatcttcaacaagtaactgggcngctgttaaagatatggagtcattacctgggatatgtttgagtagtggttctagttctaccaagtgaagagttattggtttaagngctctactgttgacccaccaaatgttatcagttactggagttatggaccagaccaactccatatccaaccccatgatttatgttcgtgtttgccaaccgg

SEQ ID NO: 30 - DNA, 301 bp, Glycine max
  misc_feature (10)..(10): n is a, c, g, or t
ctaatgcccncagttacttaaataattttaatttacctgcttttccaatgatttcagacttcccttcacctttccctgctcgttcaacttatcaagtagctgcattgccattggatgccaagattgaaatagagtgcatagcaactttataagcatcaattgtactagtacatctggctccatcagcaaactcaggttaataatgcaggaagggacatatcttagtattgaagttttacttgttttgttctttggataatggtgctcttgtgattacatttaccatcctagcgaggtctat

SEQ ID NO: 31 - DNA, 301 bp, Glycine max
  misc_feature (10)..(10): n is a, c, g, or t
ttattttttnaaaaaaatatggagcgcacgttttgtatttatggacgtgacagtctctttggttgccacgtggagnctccctcggatgcctttttgtgtagggcaaatggtgaagtgtgcaagcaacaaatagatcagacagtgcacaacacgaccgactcctcgcgcgtgatcaattttcaaataaacatgtctaacgtnggaaagaattactgtccgttagatctatcatatatctcaaaatcacttaaatccaattatcacagttgcttacgcacggtggaaacctaccttactttcc

SEQ ID NO: 32 - DNA, 301 bp, Glycine max
  misc_feature (201)..(201): n is a, c, g, or t
tgctcgcgagtgatgtagttctagtggaggactaattattagtctttgtaatgattggagaaatgagttgagtaggttttgtctgtgtaaataattatcgttatgttgaagatttcttccctaaacgcaaacttttgttcggagaaaggaagaatcagttgcttccgttcgtgcttcattaaatcatatccatccagattncattaaatattccgtcttgtttggcagaatgtcaaacaaacatgctgtgtagtaatattggcttntntgaactcattcttttntgcttttttnnaagtaa

SEQ ID NO: 33 - DNA, 797 bp, Glycine max
  misc_feature (486)..(486): n is a, c, g, or t
atgcctgcagcatggaattggtcacccagggctccagtttcttggtcatcctaatggcatgacccatggttctgatctgaagaagaattcatctcaaagtccccgttccatatttggtacaaggaatggacacttctgtgaggtatgatttttcttactacttcaatatcaatataatataaccattagaagttttacagctcttcggtattgtaattgaagattattgactgaatgtcatatacacgtaataactcaagttaaaactgcaacctgtactgcacaatgatctccaatatcccattgctcagttatgccattacctgttttaatcatctttgtgaaaatgacatcaatcatctaatcattaagttatagatagtgacaaactataatcatagcaacacgttgtttatgatttcatattatggaaatttttagtaatatttcaaaggcctgcccgtgcaaagacacatgcatattatntgggttgataccacccaaattgaactttgaaacaaattatgctcattgttgtattgacccatgctatgtataacagtatagatctgtatagagatggcttggataaaacatttttcctgtaaacctacatggtgaaattagcttttacaatgtctgtatgagcaaagagttttaaaaggtgttatgcttctactctaaaagtgaaagagcagacaattaaagtgctaagtttaaacctacttcatgctattcagcctgaaagttttcagcttcccaacaatgaaatgggagggtgcaacaaaatggggatc

SEQ ID NO: 34 - DNA, 827 bp, Glycine max
  misc_feature (207)..(207): n is a, c, g, or t
gaattggtcacccagggctccagtttcttggtcatcctaatggcatgacccatggttctgatctgaagaagaattcatctcaaagtccccgttccatatttggtacaaggaatggacacttctgtgaggtatgatttttcttactacttcaatatcaatataatataaccattagaagttttacagctcttcggtattgtaattgangattattgactgaatgtcatatacacgtaataactcaagttaaaactgcaacctgtactgcacaatgatctccaatatcccattgctcagttatgccattacctgttntaatcatctttgtgaaaatgacatcaatcatctaatcattaagttatagatagtgacaaactataatcatagcaacacgttgtttatgatttcatattatggaaatttttagtaatatttcaaaggcctgcccgtgcaaagacacatgcatattatatgggttgataccacccaaattgaactttgaaacaaattatgctcattgttgtattgacccatgctatgtataacagtatagatctgtatagagatggcttggataaaacatttttcctgtaaacctacatggtgaaattagcttttacaatgtctgtatgagcaaagagttttaaaaggtgttatgcttctactctaaaagtgaaaaaagcagacaatttaaaggtgcttaagttttaaaccttactttaatgcttattcaaaccctggaaaagttttcccagcctttccccaacaaatgaaaatgggaggggtggcaaccaaaaagggggatcccctttatggtttcaaaaaac

SEQ ID NO: 35 - DNA, 301 bp, Glycine max
  misc_feature (119)..(119): n is a, c, g, or t
taaaaaatcaacggttgaaactatgataaaatacaagcacatgtataacaagtagtagctttatacataactgagtcctagacttgcaattcaaaagtaaaagctgcaattatgatgangattaccctatagtatatactatttttttactgtagaattaaaaataccaaaacagtaaaatgaagaagatgaataagctcntagagactataccacatgcctttcatgaagcacccaaaagaagcaagaacagagagggtaataattaattaaaaaaattaataacaaagaatcacggaca

SEQ ID NO: 36 - DNA, 301 bp, Glycine max
  misc_feature (109)..(109): n is a, c, g, or t
ctccttatttatactgaattgaattggacattctgagaggcttgtattattaaattggatgacatggttttttatatgctattttgagcctgtactcaatacattgatnatggttaggtgcttttgtaacacctgagncttttggagatattaggaatgtcccacatcattatggttagntgnttttgagccacatgtncntgtaacatttattgaaccatgcctcgcctcacctctctctcatttgtttctatagacttaattaaatttttagtttttnaatttttttaaaatttaaatt

SEQ ID NO: 37 - DNA, 301 bp, Glycine max
  misc_feature (34)..(34): n is a, c, g, or t
cacaactactgactaaatgtttacatgtaagagnagtgcattcattgagttaaaagtttttgggggccatatcagtaatttacacttttattatggcctttagagagttaattacttctattgttagacacattcttaatctattgttaaaatgtcttggggcatccatgcacccaaactaatagggagaatagtanancnagccatatttataacagtgtttcccgaggacacctgatcatatgcatttttcactcaatgaaaaacaagatagtgaagccagtaagttaccacttaccag

SEQ ID NO: 38 - DNA, 301 bp, Glycine max
  misc_feature (201)..(201): n is a, c, g, or t
agtgcttcacaagcttccgggtacgccaaaatattgtcaaagcttaactcgggggattgacattgtgcttgagtcatccaaatatgagacaaagagctaatggcaccatatttgccaatggtaacagtaacagctagttctccactctcttcatgtacacctctagctaaatcacactttggactaattgcatttttctcnacttcatcgtccatcactgcaccatcactgatactcacttgtttcacaacttctgcattagaagcagaatccagaacatctccaacatcatttctcaaac

SEQ ID NO: 39 - DNA, 301 bp, Glycine max
  misc_feature (3)..(3): n is a, c, g, or t
ggnggcaaccatctccccaccaaggctgagtgatgatataatgatatatcatatatttgcttactataatacagatatgttggttgatctgcatgccttagatggttaagatacatcattgtactttagtgatttcaaagtatggaacaaaagatagttgttgtngttgttgttgtggttgtctttatacacgtaggaganttgggccagtccttcatcactctctaagcagctctttttattttctactttcctttttctcttagcttagcacaccatatcactgaggttgcttaaatgc

SEQ ID NO: 40 - DNA, 332 bp, Glycine max
  misc_feature (149)..(149): n is a, c, g, or t
tttgcttactataatacagatatgttggttgatctgcatgccttagatggttaagatacatcattgtactttagtgatttcaaagtatggaacaaaagatagttgttgttgtcgttgttgttgtggttgtctttatacacgtaggaganttgggccagtccttcatcactctctaagcagctctttttattttctactttcctttttctcttagcttagcacaccatatcactgaggttgcttaaatgcaaaactggctcttatttgttgcatacttgacaaactcgatggaaattccaattgaaatttatcacgccttgttatttgactgg

SEQ ID NO: 41 - DNA, 301 bp, Glycine max
  misc_feature (34)..(34): n is a, c, g, or t
tcaaatccttaaccacatatttatgagataaaantatataccaattacattagtgtgaagaaagttattagaagactttgcantcgtagtggaagactanattagagtatcaactgctctctctattttgctgggttgctcagctgatagcctcctctattatggtttggcttgtgtcagaatgatggcattttgattgtnctttcatttggttaacagatcacatttatttcaatggttatttttaaattttctctttattatgttcncccctttgttggaaatatttctttttatttat

SEQ ID NO: 42 - DNA, 470 bp, Glycine max
  misc_feature (371)..(371): n is a, c, g, or t
agactttgaggcatttcttttgttacaggagatttggtttcacaagaatgcttcaccttatgttctatatctgaaactgaaatgcatggcataggtctatgtagatagatgggagattggttgatgacatagtcactgcattcatgtgcttgcagttataacactctagtcctaactactgtgccacctttttaagtttcatcatgttcctaatgacatgctactagctaggtcgttatttggaactctagtcagtgtagagtggatctgatgggattcatgtgaaaggtaaaggtaggacgatctcttccattggcttttataagtaaattggtgttttagttggcaaggaatagtaggttaactaaatnaggatagcaactatttattcgagtgcttttcaggttaagggaattgtggattattttggttggctccatctattttgnaattttatttcatatggggtt

SEQ ID NO: 43 - DNA, 301 bp, Glycine max
  misc_feature (13)..(13): n is a, c, g, or t
agtcaatgtaatnaacgtctagaggcagttnaaaagattctatttctttacatgaggcttatagtttcaaattttgagctcnacacatttcnttacaacttgaattgtccccgttagcaaaatacatatcaacatttcttcaccttgacttgaagggtctcaagctatttgcaaacattcaccaagtaaagtctcagtagncaaagggccttgacaacatgtccattaattttcagcaatggctatctttccatggagaacaacttatctttaatatcatcttgagtttctttcaacctaa

SEQ ID NO: 44 - DNA, 301 bp, Glycine max
  misc_feature (123)..(123): n is a, c, g, or t
ataagatgccgaatgcatcatgacaattgagaaagatacatatacttctctcataccacaccccataacattattcttgtttaatttcttatacaataaataaaatgataagagcagggatangtttgagataaaataacacaaatattcttgaacagccatgaacctttttgacatctaagagtggaagaaagaaaacanaaaaaaaagatataataaaaaatatgatgtgataaaaaaataaagaaaatagtgagtgctaatgatttttttttttttacttttatagaaatatatttgt

SEQ ID NO: 45 - DNA, 301 bp, Glycine max
  misc_feature (171)..(171): n is a, c, g, or t
gcaaacgttcaactctaaccatttcttcgagaggacagaagttctcacagcttccttgaagggaagaagtgaaacaatagttataagcaattcaattggcaacgaattgatgctcattaggctctagctataagatatggtgattgataataaaggtgtggaagggtactnaaattttcttcaaatattccaatggggtanaaatgtgtgtgtatgaaatggccaatgagtgcaaccttatgcaacagacgttgcntcagtnagaaaacaatgttgcatacttctatatggttttttgtat

SEQ ID NO: 46 - DNA, 301 bp, Glycine max
  misc_feature (103)..(103): n is a, c, g, or t
tatataggaaaaacaaattccattcacgtaattgatactcatagataggtcatttattgaacctagttcaatggtgagacgctctaacataggacaactattnaggaataatgagattcgagaaaactcattatgttgcaaggccatcttcatgatcgaatgtcgtacatccatgtcaggttccatatgtagctgttcggnaccagtagaaattaccttaaaatatcacacgcaatacgttagttcaaacaatttagataagatattgnaattaatccatatttcagttcattcaaacaga

SEQ ID NO: 47 - DNA, 301 bp, Glycine max
  misc_feature (201)..(201): n is a, c, g, or t
gcgaatgtgtcagtgtcagagccgtgatacaaactctcacggactgagcactatttttttttatgtgatttaagaatgtcgttgtgatgattcggctcaatgtgaatgtgatgcccctctgagatcctcagatgtttagtttagtgcaaatcgaagttactgctgcagaatccgaaaaatttactaggttgttatcttaanttagaaaagatcaagcataaaaatagaaaagatatggtaaactttttaaggatcgatgactnggaatctgatttgggaaatggtgagatggaggttacat

SEQ ID NO: 48 - DNA, 301 bp, Glycine max
  misc_feature (33)..(33): n is a, c, g, or t
gaaactgaaaaatatattagaggaattattttnaaaaaaaaaccacttttgggctctgggccttggcccgacttaattggatnttgcactatatgaaagagagttgggtgggttaatctaaacgagtacatggagtgaacaatattttccctcctctaaggtcccaatttttcttaaaccctaaaacactctcactgaagnagctgctgcagaagagaatcccgatttttccaaaatgtcgatgtcgaagagttccaagatgcttcagtacatcaactaccgtatgcgtgtcaccattcag

SEQ ID NO: 49 - DNA, 301 bp, Glycine max
  misc_feature (201)..(201): n is a, c, g, or t
atgactggcttcctgtaacaagcaaatataattaatgctaaataccagtacgacttctagaaatgataaatgacgaaacaataataattcattttaattagtattataaaaacaaaaattcgaaatagctcatacccaatgaatccaccagaaatgttgaactcgaagtagtagtttaataatttggttcaaaagaagaancatgatagtactcagtagagtccaaaatggaatgctgcatttgtagtactaccaagttaagctagctggcatagcagtgtagtggcagtcaacttcatgc

SEQ ID NO: 50 - DNA, 301 bp, Glycine max
  misc_feature (112)..(112): n is a, c, g, or t
ttttgtaaggtcagttatggtatcattttgtttatagccgtgaacatgggtaattactaactagctagcactaaacttcattcagagagaagcaaaaatgaaacagatatgnctatagtctttcctgtgtagactaatttttctaagttgaatggttgttgctctgatgattcatgtgtcccttatgaacttctaacatanaccttgctatagatgaacaccatatcactttgtttgaataatactgaaggaagattataaagacagctataaaatgtagaacatgcttatatttatgaac

SEQ ID NO: 51 - DNA, 775 bp, Glycine max
  misc_feature (220)..(220): n is a, c, g, or t
gaaaccataatccattatgaatttaaaaattgtagtaaaagggaaagattaccatttatgtctacctctacaagacctccactagagttctgttatcaataaaattcatcattctttcctttttatctgatccagttctatcagttttgttcttcatttatgcaaaagctaaaagtaaatatttttcccttgcacagattttaaacctccatcagggganggcaaaagaacttatctcatactggccatcatcatagtagctggtgttttagtggttgttcttctggtgctggttctcttgaggaggatgggatggttaggaggcaaagatccagtgtataaaggtatggaatatgcccccaagttcttccaattcttttagttagaacaaatatttccatttgtttccancacaacatcttttctgtatccagnagtatttgcctgagcttcttcaacaatttgtttgaataaatatttctacaaactttattcaaatgtcataaaactgaagtgcaacaatttacttctatataagatttgtaatttatgatatttctccatttcaccaaagtcaaggctttaagagaacatacgagagtatacttgcctgccctatatatttatatactagatagaaatgaaatgaactatgtggcagatattgtagagaaagttataaactttcttgttgaatccaaatttccaatcttttgttttttattttcagagctcaggggcatagatctgcaggtcgactctagagcaaagacgg

SEQ ID NO: 52 - DNA, 493 bp, Glycine max
  misc_feature (361)..(361): n is a, c, g, or t
tgaatgagggggaatattagagattagagatactgagataataaaactaatcttggaattcctttgggatgctacctcttcctgtgggttactacaatgtacttgtgaatgatactctttctcttattaggattcaacattgtttattgttattgctctgttatatacttatattcctttatgcagatttgaccctttttcatttaattttttttataattaatgcagggtcttgatatttatgactcaaagcaaataagagacaaacagataacacgggttattggaaaggtgtgcttatttggttctttcttctcctttcgctgttctatctctgaaaacatgcaaattatttagttanttgaatgattctagccctggttttattgattgtatcttggtggattttgtccactatcctctcaaccctatgattatttgttatgtgtcctactgctttattgccatatcatattttcactcctttttattt

SEQ ID NO: 53 - DNA, 301 bp, Glycine max
  misc_feature (20)..(20): n is a, c, g, or t
aaattcattcaatctatcantaacgattaaattcaaaatataagcagaacatcacatatacatgaactctcaacttattaaattattantattttattaaaaacactaaagaaaataatgcatttccacaccttccttnattgtcaagttattttttttagtttttaatatatatataataaattttatattttaattacnagttcagattgcttataaagtatgaatggcgcagctaggtaagttgcgcgcggcatgaattgaagagtgaaaggaagatgagggtggcngaggaggagaa

SEQ ID NO: 54 - DNA, 301 bp, Glycine max
  misc_feature (108)..(108): n is a, c, g, or t
ttcattgaacagattctctctttcgtttcgaaagctgaccaggtatcggcgatggcggcgaacttggcgagctcattgtgcttcaaggatgatgatggggccgagggntgttgtttgggctgcggttgagncattgaagaagcagcagcatcgatgaagaacctagtagtagcgttaaggagaggctgaaaacgatggtgnagaatgcggtggatggagatgcgattgaggtttcgaagatggttgaaagccatggacatggcaatggcaatggttccgcgctcaaaatcttcgtctcttt

SEQ ID NO: 55 - DNA, 301 bp, Glycine max
  misc_feature (201)..(201): n is a, c, g, or t
tgacccataaaatataaatgtttgtacataagtatatattcatgatcattttagtaagatgccatgcccaacagtttgttgtggtaccaatatgttcttgtactttttttttatgtgaagacaaaatgctcattttctgaaagttcaaaatatgattattttatttgtggatgatgggcaattttgaatatagaatatacngattgacaaccaacagtgggttgttatttgtagagatgcccagcaaaatattgactggaacttgcatggttttctgttcctctagatgattgaaggttgt

SEQ ID NO: 56 - DNA, 301 bp, Glycine max
  misc_feature (5)..(5): n is a, c, g, or t
atganaaatccangtgttggaagtgaaagccacntgttggactaggtatgtgtggaccaaattgtcaattggagtatgtgtgtgtttttaccttttattaattcaccatttatcctcttcctttttacattttgtgagaaagatgttgaattgtgggtgctaagtttgctttgctctttcctgcctttgcatcatcggtgnctgtgacgacttaacgacagatggtggtgtttggttggtggccnctgttttngtttgattttttcctgtgcgttgacggtgaaagacaactttataaggt

SEQ ID NO: 57 - DNA, 301 bp, Glycine max
  misc_feature (141)..(141): n is a, c, g, or t
tatggtacgtataatggttttggacgttccgtcgttgtgtgagacacctgattttatattctgaaagtgttggtttgtgggtagaatgacttggatgtatcggtcaacataagttagcgactcatgttttggacatgtgcngataatttcgagtgtctattagaacccaacctaggaggtctatatagaaccaatggaaancggaaaacaatgtcagtttgtggttacgctggccgaaatttgaaagtggaagtaaattagtcaagtatttaacttggctagccatgaaggttactcgatg

SEQ ID NO: 58 - DNA, 301 bp, Glycine max
  misc_feature (100)..(100): n is a, c, g, or t
agggtgcatgtgaggtcacaccgttgtgtcctaacacattgtaacacctaattcaaaagaatgtttaatgaagataatttaaatattaactaagtcattnaaatcaacaaggtactggaaagtcacgagaaggaagaaaatttgcaactgaaaatacaaggtagacattgaagaattaagattatgattaggacgtctcangggaagcctatgcagagacgtcaacataaacctggtcaaattctgtaataaaaaaaataatgaatcatactaacactccatggaaanggaatttaatgat

SEQ ID NO: 59 - DNA, 301 bp, Glycine max
  misc_feature (77)..(77): n is a, c, g, or t
taaaaaattaatttaaacttatatgtaacctttgtcaaaacattatatataagttaattttaatttgtgtgtaatgnttatttcatttttacttattattttttctcttataattaccagaaaatttatctaaataggtacgtgtattaggaataagctcttcggctgcatgatttcaacataaattaagaatatacccgntcacttgagagaaaaaagtcacatagtagagctgtccattccctccagcatacatacagaaattgcattaagtagtaatccctttgtcctaaatctttaa

SEQ ID NO: 60 - DNA, 301 bp, Glycine max
  misc_feature (7)..(7): n is a, c, g, or t
actattnaatcatgtttataattataaatttacatgtgcacagaaaagtaatgttatttatntattgtaacattgtatttagaatattttttataacattatttgttccgaactgttactcgacccatcttggtctatttgattagagtagacatgttgacccaaaaactatagaagtccaattcatcactacnnaacaangtttaatgttcctcaccacttcctaatgttgcacaagagtcaagagctcatatatacgaacgtgaaatgctgaaanatatgacataaaactgtcagcaat

SEQ ID NO: 61 - DNA, 301 bp, Glycine max
  misc_feature (10)..(10): n is a, c, g, or t
gtaagtcttnaaaatcaacttgcggatcatntatatcaactgnggatcaactaaaacctatgcggatcaacctgaatgagttgggtgcacaaaantntttttgaaaatatacaagggtatttttgtnttttcatattaagtgctgggtgcaccagcaataatgctggntgcacctagcaacaccctttttaatattaactntttttgggctttgatttgcctaattgtgcaagacacagccatgtaaagcccaaaaagaaaaattgccatcaaaaccaacttcctccaatcagaccttttt

SEQ ID NO: 62 - DNA, 866 bp, Glycine max
  misc_feature (353)..(354): n is a, c, g, or t
aaatgtcatgtataaaaaaactcttggccaagtgactaataataagtattttcttactgtcatgatatgaaaaatatatagattttgtgaataattaatcctgatgagaaaatttgattaatcttttaagatattttcctctaaccacaagactaataagtgtatgatctaaatgtgcacactctataactacaaacaacgctcattttaatagtttttaaattctatttatctccttatcctgttagatatacatttacttctttatcttttaagctgtcaaatttggacaatcgttcaacctaacataagaagtcgaaaagtctaagacagggatagaacgaccatgcgannttggtggtcttnatgggccgatggttagcaccttctctgggtgctttgcatcccattttgaaaaatatttcataaacatccataatactatcttacacttagacagagaattaaaaaagaganaaaattataaatatgatagaatttataacatgaagaaagaaatagaaaaaaaataaaagatattgatggaatatttagattatgtatgtatatcattactctatctatttaaagatattcaaagctaatcttctaatattttttagatgagtgaacaaaataaagatcttctactctatcgaaattgattttttaaggggattactttattgccttcaaccatattgactggtaataggttaatgttaacctataaaaggtgtgcttttgcgggaggaacgtgatggaaactctatttggttaaaaaagatattccagttcggtcacttaagattcataagggccgattattttttataaattggcttgtttaaaaaaatttttttttt

SEQ ID NO: 63 - DNA, 1394 bp, Glycine max
  misc_feature (1026)..(1026): n is a, c, g, or t
ctctgtacccggggtatcctcttggatcgacctgcagcttgcatatgcagtaaatgccaaacaataaacaagcgaccagatcattcaaatgcattgtctcaatttcggatttacctcattttctaggtcattctttttaatgtttttaatgtatgaccttaaattaatgatcaatgtaaatcaacaaacaatgttacagtacattttagagccaatcttgtggctcttttttttctttttttttcttcaattctattatacgggctgcaaattgcaatgcaacattataagagtgttccaccaggttttctgataggttagtaacaacctaaacaagtctctttgcatcacactttccaccattagcattttaactattctaaagtaagaagtaggaaaaaacaagagttacaacttacaagtggttgtacattctaaaagctagcctccacataaaagttaatcttatccagtatagctagctattagatagagcttccaaaaggtagctacagtaacttcctccacatggaaggccaatgtggctccatgaggggtgaaggtgggcacatgtccgttcaaatttttaaaatcatatgaatactgtgatgtcgtattcggtccaatgctcaaactatttgtttggggtttgattccaataaaacaatgaggtatttttcttttttcaaaataaaaaacctaaatagaaggaaattatgcaagcaccaaacctactttcaaggataaattgttactgtgcctaggtgaggaaaatttggtagcatttgacccatttgataaaattttaaatggaatccagttgttaaagaaactcgtgaggaatctgtttttataatggccaaaggtggttaaaaacactatggttaaacaatgaaactcctttgtatccggtgtgtataacccgcataaaaacactttacattatccacatttttactcgtacagtagtacacgtaccagtctgatttatttatttctacttggaaaatagttattgcaggaaantttttttaaaaaaaaaaattatcttgagcaaaggaatgtgtggattgaattacaagataggactcaaattcggttgtacagtttattgataactgaaaccaatgtttacaaggtttgcccagagaaagctggctctttctggggaagcactaactcatgtcatcatcatcgtcttacacaaagcaccaccagagactgctacttctccactatgaagaaccttttcttattctccacaaggattgctccacctggccaaactattcacaagacaacattcttctagaacaaccttgtcatgccatactgaacaggtccaacctttctatgagcagtggtactaatcccaccatattgatgagcctttg

SEQ ID NO: 64 - DNA, 301 bp, Glycine max
  misc_feature (10)..(10): n is a, c, g, or t
cggattgggncatggngattgtatgagagatttggttgtgttgggcatatgggactccatgtgctcagacccactcggatccctctgttcttgtcacattcacacactacaccacccattttttttaatgccacactttttttcaatccttcataattcttttaattttgttctggagggtaatgggggtccgtaccaccntttcttgtacgaaaattgaaatcaaattgttacataggggccttgctttgctttggcacatatcattgatgagagagctattataattcaatatcatgca

SEQ ID NO: 65 - DNA, 301 bp, Glycine max
  misc_feature (67)..(67): n is a, c, g, or t
tggcagggaatgaagggattgtgcatgaagacaagtggagaaaatgctattgatagatatgaaatgncatattgtcttgtccacttatttttaggttgccttattttgtgcttctttcttcttcttcttcttttgtttttttgctcttattggatatgacatttatgattgtgatattaactgaatgatagtgtcaagcantagcatatctactttacgtacactgcttccttccttaaagtaactaggacttgactaggaatcttgggcttacataacttacaccaaatttgtatttcac

SEQ ID NO: 66 - DNA, 301 bp, Glycine max
  misc_feature (97)..(97): n is a, c, g, or t
aactaattaattaaattaagtaaaactatttcaaagaatcaacattggttgattttcactagaaggtctgtgttagagacgttagaaaggaattcangttgaatgctatatatggtaagccacacgatttttactgcatattcaacatttgagacgctatcctagcatctaattttgagatcttctcatatacaacgtctnagacatctgattagttttggatcctctcgcattgcatcaagcatgaattcctttttagtgtcaatagatttggacttattatgggtcaaaacctntaaat

SEQ ID NO: 67 - DNA, 301 bp, Glycine max
  misc_feature (79)..(79): n is a, c, g, or t
tgaaaatgttttttgcataaaggtaccttacactcggagaagaaggtactctactctgtgatggctgccacgagtgggnttgtgagcaagtaaatttgtaagtgaagttaactttaggttaacctttttagttgttaataaaaattcaaccctttcactcatggaggcctgagttgaacatgccttacttcaaactaagangcttaaantagcatgcatgagacatttcatagtacattagttcacacagaaatttatgcagtctagttaagaatacattttttgttctcaacatttcacg

SEQ ID NO: 68 - DNA, 301 bp, Glycine max
  misc_feature (65)..(65): n is a, c, g, or t
acaagagatgaaacaaagaaaatggaagaagaagagttctcaagtgaatattttgcttctttttngtgctcttgttgccaacttcttccaaaagatacaatatatcctctttatattcttaaaaatgctacacaattatgcttttctaaaatcactgtgccctaagcctacatatgagcgcttagcgcacatgtaaagtanaattggcttaagtnatcatgcgtcaagcctccagtcgcgtgcttagcgcccatacatgatacaagcagcttctagcttggcttctctcactgagcgtgca

SEQ ID NO: 69 - DNA, 301 bp, Glycine max
  misc_feature (35)..(35): n is a, c, g, or t
tttatttattttctacttaaaaattaaatacgcanttttttttgtgtgtaactattcaaacacgggggcctaaaatcctaaatgcatgttttccgcagatcaaatcatacttaaaangnaaaaaaaaacaagttgcttaaaatttcagtgctctcatactcatttgcttagcaagttgcctcgctctgacctttttgagangagttactgtgttgattatttttctgcnatgttgtactaacttgtgtttattgttttattatttgaacataaaaaatttaatattagatatattattatn

SEQ ID NO: 70 - DNA, 301 bp, Glycine max
  misc_feature (2)..(2): n is a, c, g, or t
tntagataatttttgacttatagataataactaagagcttgcagacattgtcagagataaacacttgcttatacatagaaggtanaatataatcgtatcttatagataatgtgtgactttatagataatttaatatctttcaatcaataaaataatacattgatttaaatattaataggttagagatacctgtttgntganaagtttaacagcggatgagacatctacactcttgtaacaagaccgatgtgaaaagttgacacatatctctcattgacaaatagaatgccacacaaatttt

SEQ ID NO: 71 - DNA, 301 bp, Glycine max
  misc_feature (119)..(119): n is a, c, g, or t
tcaaattgtattcaaatctaaatcgagttgattttttctcaatctaattcaagaaaaaagagtgtactattttataaaactaatttggttaacatattgtttctacaaaatcaatttgnttaancaaattaattttcattcaaatatgtttatttgaaaaaaatagctcaaaactagtttaaaaaccaattcnaacctggntgggctcttaaaacatacccaattgtcttacacgtggcttgtttctattttttnataatgtcttttgaattttattactttaaaaagataatattttaaa

SEQ ID NO: 72 - DNA, 301 bp, Glycine max
  misc_feature (201)..(201): n is a, c, g, or t
agcaaccaaagcaagagtttaacaaatcactaattgcatcttaatcatgattgaaggtaatctcagactacaaaatttcctgtaagctaaatttgaagatgaaagactcccctctcaggctttattggcaatggcatacaataaaacagaatccaactaatacaagaagcagcatacataattcagcagtttcaaaaccancacaataaaaaaggcatgccgaatggattcacagttcattgggaaagtaaaataacctgatgaatgttgataagcacaacaatttctaatgaaaaaaaga

SEQ ID NO: 73 - DNA, 301 bp, Glycine max
  misc_feature (11)..(11): n is a, c, g, or t
ttaataacagntcattacttgtcatatacgtaaagatnataggaccatgatcatctcagaccatctaataatttatctcaccaactggcatcaaaacacacactaaatgggagggtcacagcttattcatgtaacaatgtcaaatacctgtccaccagtatcagaatctcgaccaagctccatgttttctcctcgaaccanaccatgcaaactgtcaaacgacaagggtcatagtcaaacaatcaatataccttagtaatacacaatcaagaaatttggctaaacacatttattgaatgca

SEQ ID NO: 74 - DNA, 301 bp, Glycine max
  misc_feature (66)..(66): n is a, c, g, or t
attaaaagattgaaaatttaaaccatcttgagacttttgtaaatcgttaaaatatccatactcgtngaaaattccatagcacgtgctcaatgctctgcgtatcatatactacanaatacattttattttttgtaaaaatttccatacattaaaataattgatattgtttatttccactattttattttccttcttgtcatngggttggtacatcctgtaaatacaaaaattagacagcccatatgtacccagaaacaaaaatatagtccataacagtgagccacttattagggcagcccat

SEQ ID NO: 75 - DNA, 301 bp, Glycine max
  misc_feature (102)..(102): n is a, c, g, or t
gtttgtttacatcagaaggaaattatgttgttgcttgccttggattcatatatgctcttcttcatccatcgcacgacaatagaaattattgtttgtcctctnaacgccatagtttgatatgtagatgatagattgtattgtgattatattgtgggtgcttctctcgtagaattggtttactttaccgacataattgtgtancgaataccctgtggctttagcttattatgcaaaatggccagccaaaccttggtgtttgaattttagtttggaaattattagcacaaacaggtttattttt

SEQ ID NO: 76 - DNA, 121 bp, Glycine max
  misc_feature (61)..(61): n is a, c, g, or t
aatttttactcaattttcaaactttcatgccagcctattttcctcccatacaactactacngctaaacacattatctttgacgaacaaatcatattacaacacccaaattctaccatgaat

SEQ ID NO: 77 - DNA, 301 bp, Glycine max
  misc_feature (201)..(201): n is a, c, g, or t
tataatagcaatgtagacaaggagaagaatatacaaaattataataccaacttaattgatcaaaattagaaagaatacaatgcaggaatgaaataccttgatgttatggtcttttttgcttgaatgtctgcctttagctttcttgtgctcttgagatgcgctaacaaggagaaacatgcttaattgaaagaggaaagcacngagtttttgntgaactcccattcttcaatgctagtgaataagacttcaaggcagaaacactgacattctggaatttatatgtgtttcttctcnagctggc

SEQ ID NO: 78 - DNA, 22 bp, Glycine max
cccattgctcagttatgccatt

SEQ ID NO: 79 - DNA, 28 bp, Glycine max
aacgtgttgctatgattatagtttgtca

SEQ ID NO: 80 - DNA, 16 bp, Glycine max
acctgttctaatcatc

SEQ ID NO: 81 - DNA, 18 bp, Glycine max
cctgttttaatcatcttt

SEQ ID NO: 82 - DNA, 22 bp, Glycine max
atgatcaagcgttgaaggttca

SEQ ID NO: 83 - DNA, 23 bp, Glycine max
cttgacaagaaacccaaaaacca

SEQ ID NO: 84 - DNA, 18 bp, Glycine max
atccttcgatatcagtca

SEQ ID NO: 85 - DNA, 14 bp, Glycine max
atccttcgaactcc

SEQ ID NO: 86 - DNA, 25 bp, Glycine max
gcaattgtcgatcccgtgttaaata

SEQ ID NO: 87 - DNA, 40 bp, Glycine max
gttttagtttgatcttattttaaaagttttactttagctt

SEQ ID NO: 88 - DNA, 19 bp, Glycine max
tagattaaattagtctttt

SEQ ID NO: 89 - DNA, 16 bp, Glycine max
attaaattggtctttt

SEQ ID NO: 90 - DNA, 19 bp, Glycine max
tgcccgtgcaaagacacat

SEQ ID NO: 91 - DNA, 27 bp, Glycine max
aatgagcataatttgtttcaaagttca

SEQ ID NO: 92 - DNA, 15 bp, Glycine max
caacccataatatgc

SEQ ID NO: 93 - DNA, 18 bp, Glycine max
tggtatcaacccatataa

SEQ ID NO: 94 - DNA, 23 bp, Glycine max
tggtgttttagttggcaaggaat

SEQ ID NO: 95 - DNA, 23 bp, Glycine max
ccttaacctgaaaagcactcgaa

SEQ ID NO: 96 - DNA, 18 bp, Glycine max
ttgctatcctcatttagt

SEQ ID NO: 97 - DNA, 17 bp, Glycine max
ttgctatcctaatttag

SEQ ID NO: 98 - DNA, 24 bp, Glycine max
ttcccttgcacagattttaaacct

SEQ ID NO: 99 - DNA, 26 bp, Glycine max
gatgatggccagtatgagataagttc

SEQ ID NO: 100 - DNA, 15 bp, Glycine max
ttgccttcccctgat

SEQ ID NO: 101 - DNA, 15 bp, Glycine max
ttgccatcccctgat

SEQ ID NO: 102 - DNA, 25 bp, Glycine max
gctgttctatctctgaaaacatgca

SEQ ID NO: 103 - DNA, 26 bp, Glycine max
ccaccaagatacaatcaataaaacca

SEQ ID NO: 104 - DNA, 19 bp, Glycine max
cattcaagtaactaaataa

SEQ ID NO: 105 - DNA, 17 bp, Glycine max
agaatcattcaaataac

SEQ ID NO: 106 - DNA, 20 bp, Glycine max
gggatagaacgaccatgcga

SEQ ID NO: 107 - DNA, 20 bp, Glycine max
gcaaagcacccagagaaggt

SEQ ID NO: 108 - DNA, 14 bp, Glycine max
tggtcttcatgggc

SEQ ID NO: 109 - DNA, 16 bp, Glycine max
tggtctttatgggccg

SEQ ID NO: 110 - DNA, 28 bp, Glycine max
tcgtacagtagtacacgtaccagtctga

SEQ ID NO: 111 - DNA, 26 bp, Glycine max
cacacattcctttgctcaagataatt

SEQ ID NO: 112 - DNA, 13 bp, Glycine max
tgcaggaaaattt

SEQ ID NO: 113 - DNA, 13 bp, Glycine max
tgcaggaaatttt

(19) United States
(12) Patent Application Publication
(10) Pub. No.: US 2016/0050864 A1
(43) Pub. Date: Feb. 25, 2016

(54) Methods for Producing Soybean Plants with Improved Fungi Resistance and Compositions Thereof

(71) Applicant: MONSANTO TECHNOLOGY LLC, St. Louis, MO (US)

(72) Inventors: George J. Baley, Webster Groves, MO (US); Vergel C. Concibido, Maryland Heights, MO (US); Jennifer L. Yates, Ballwin, MO (US); Xianghai Ye, O'Fallon, MO (US)

(73) Assignee: MONSANTO TECHNOLOGY LLC, St. Louis, MO (US)

(21) Appl. No.: 14/783,290

(22) PCT Filed: Apr. 9, 2014
(86) PCT No.: PCT/US14/33424

§ 371 Date: Oct. 8, 2015

Related U.S. Application Data
(60) Provisional application No. 61/810,439, filed on Apr. 10, 2013.

(51) Int. Cl. A01H 1/04

(52) U.S. Cl. CPC A01H 1/04; C12Q 1/6895; C12Q 2600/158; C12Q 2600/172; C12Q 2600/13

(57) ABSTRACT

The present invention provides methods and compositions for the identification and selection of loci modulating phenotypic expression of a stem canker resistance trait in plant breeding. In addition, methods are provided for screening germplasm entries for the performance and expression of this trait.

### INCORPORATION OF SEQUENCE LISTING

A sequence listing containing the file named “59630_ORD_ST25.txt” which is 67,400 bytes (measured in MS-Windows®) and created on Apr. 7, 2014, comprises 113 nucleotide sequences, is provided herewith via the USPTO's EFS system and is herein incorporated by reference in its entirety.

### BACKGROUND OF INVENTION

Soybean, Glycine max (L.) Merr., is a major economic crop worldwide and is a primary source of vegetable oil and protein (Sinclair and Backman, Compendium of Soybean Diseases, 3^{rd} Ed. APS Press, St. Paul. Minn., p. 106, (1989)). The growing demand for low cholesterol and high fiber diets has also increased soybean's importance as a health food.

Soybean varieties grown in the United States have a narrow genetic base. Six introductions, ‘Mandarin,’‘Manchu,’‘Mandarin’ (Ottawa), “Richland,’‘AK’ (Harrow), and ‘Mukden,’ contributed nearly 70% of the germplasm represented in 136 cultivar releases. To date, modern day cultivars can be traced back from these six soybean strains from China. In a study conducted by Cox et al., Crop Sci. 25:529-532 (1988), the soybean germplasm is comprised of 90% adapted materials, 9% unadapted, and only 1% from exotic species. The genetic base of cultivated soybean could be widened through exotic species. In addition, exotic species may possess such key traits as disease, stress, and insect resistance.

Soybean stem canker is caused by the seed-borne fungi Diaporthe phaseolorum and results in reductions in yield. D. phaseolorum often infects the plant surface (often the leaf) via ascospores, which release phytotoxin and grow hyphae. The hyphae give rise to perithecia in the reproductive stage of the D. phaseolorum lifecycle. Infection occurs in the early vegetative stages, but symptoms are delayed until the early reprocutive stages. Symptoms include dark lesions on the stem exterior, interveinal chlorosis, and foliar necrosis. It is estimated that up to 50-80% of a farmer's yield can be lost due to stem canker (Kilen et al. (1985) Crop Science. 25(1):50-51).

There is a need in the art of plant breeding to identify additional markers linked to quantitative trait loci associated with stem canker resistance in soybean. There is in particular a need for numerous markers that are closely associated with stem canker resistance QTLs in soybean that permit introgression and maintenance of the stem canker resistance QTL in the absence of extraneous linked DNA from the source germplasm containing the QTL. Additionally, there is a need for rapid, cost-efficient method to assay the absence or presence of stem canker resistance loci in soybean.

### SUMMARY OF INVENTION

In certain embodiments, the present invention provides methods for producing stem canker resistant soybean plants by employing polymorphic nucleic acids useful for identifying or producing stem canker resistant soybean plants. In certain embodiments, the present invention further relates to methods of determining the presence or absence of quantitative trait loci conferring stem canker resistance in soybean plants, including but not limited to exotic germplasm, populations, lines, elite lines, cultivars and varieties. In certain embodiments, the invention relates to methods that provide for identification of molecular markers associated with stem canker resistance quantitative trait loci (QTL). In certain embodiments, the present invention relates to the use of molecular markers to screen and select for stem canker resistance within soybean plants, including but not limited to exotic germplasm, populations, lines, elite lines, and varieties. In certain embodiments, the present invention relates to the use of molecular markers to maintain stem canker resistance in soybean plants.

Methods of creating a population of soybean plants with enhanced stem canker resistance are provided. In certain embodiments, the method of creating a soybean plant with enhanced stem canker resistance can comprise: providing a first population of soybean plants, detecting the presence of a genetic marker that is genetically linked to a stem canker resistance locus on linkage group B2 by 20cM or less in the first population, selecting one or more soybean plants containing said marker from the first population of soybean plants, and producing a population of offspring from at least one of said selected soybean plants.

In certain embodiments of the invention, the genetic marker detected is genetically linked to the stem canker resistance locus by less than 15cM. In certain embodiments of the methods, the genetic marker is linked to the stem canker resistance locus by less than 10cM.

In certain embodiments of the invention, the genetic marker is located within a chromosome interval comprising and flanked by Glyma14g00220 and Glyma14g05460. In certain embodiments of the invention, the genetic marker is located within a chromosome interval comprising and flanked by Glyma14g02010 and Glyma14g03380. In certain embodiments of the invention, the genetic marker detected is located within a chromosome interval comprising and flanked by SEQ ID NO. 1 and SEQ ID NO. 77. In certain embodiments of the invention, the genetic marker detected is located within a chromosome interval comprising and flanked by SEQ ID NO. 6 and SEQ ID NO. 71. In certain embodiments of the invention, the genetic marker detected is located within a chromosome interval comprising and flanked by SEQ TD NO. 24 and SEQ ID NO. 64. In certain embodiments of the invention, the genetic marker detected is selected from at least one of the group consisting of SEQ ID NOs. 1-77.

In certain embodiments of the invention, a portion of the aforementioned stem canker is caused by Diaporthe phaseolorum.

Also provided is a method for creating a population of soybean plants comprising at least one allele associated with enhanced stem canker resistance comprising at least one sequence sleeted from the group consisting of SEQ ID NO: 1 to 77. In certain embodiments of the invention, the method comprises the steps of genotyping a first population of soybean plants, said population containing at least one allele associated with enhanced stem canker resistance, the at least one allele associated with enhanced stem canker resistance comprising at least on sequence selected from the group consisting of SEQ ID NO: 1 to 77; selecting from said first population one or more identified soybean plants containing said at least one allele associated with enhanced stem canker resistance comprising at least one sequence selected from the group consisting of SEQ ID NO: 1 to 77; and producing a population of soybean plants comprising at least one allele associated with enhanced stein canker resistance comprising at least one sequence selected from the group consisting of SEQ ID NO: 1 to 77.

In certain embodiments of the invention, methods of creating a population of soybean plants with enhanced stem canker resistance may include introgressing a stem canker resistance locus or loci into a population of plants. In certain embodiments of the invention, methods of creating a population of soybean plants with enhanced stem canker resistance may include maintaining a stem canker resistance locus or loci in a population of plants.

In certain embodiments of any of the aforementioned methods, the identified or the selected plant is stem canker resistant.

Further areas of applicability will become apparent from the description provided herein. It should be understood that the description and specific examples are intended for purposes of illustration only and are not intended to limit the scope of the present disclosure.

### DETAILED DESCRIPTION OF THE INVENTION

In accordance with the present invention, Applicants have discovered genomic regions, associated markers, and associated methods for identifying and associating genotypes that confer a stem canker resistance phenotype. For example, in one embodiment, the invention comprises a method for screening a population of plants for the presence of molecular markers linked to a stem canker resistance locus. In another embodiment, the invention comprises a method for selecting plants containing a molecular markers linked to a stem canker resistance locus. In another embodiment the invention comprises a method for creating a population of plants with enhanced resistance to stem canker using molecular markers. In another embodiment, the invention comprises a method for maintaining the presence of a stem canker resistance locus in a population of plants using molecular markers.

The use of markers to infer a phenotype of interest results in the economization of a breeding program by substituting costly, time-intensive phenotyping assays with genotyping assays. Further, breeding programs can be designed to explicitly drive the frequency of specific, favorable phenotypes by targeting particular genotypes (U.S. Pat. No. 6,399,855). Fidelity of these associations may be monitored continuously to ensure maintained predictive ability and, thus, informed breeding decisions (U.S. Pat. No. 8,039,686). In this case, costly, time-intensive phenotyping assays required for determining if a plant or plants contains a genomic region associated with a stem canker resistance phenotype can be supplanted by genotypic assays that provide for identification of a plant or plants that contain the desired genomic region.

### I. DEFINITIONS

Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “adjacent”, when used to describe a nucleic acid molecule that hybridizes to DNA containing a polymorphism, refers to a nucleic acid that hybridizes to DNA sequences that directly abut the polymorphic nucleotide base position. For example, a nucleic acid molecule that can be used in a single base extension assay is “adjacent” to the polymorphism.

As used herein, an “allele” refers to one of two or more alternative forms of a genomic sequence at a given locus on a chromosome. When all the alleles present at a given locus on a chromosome are the same, that plant is homozygous at that locus. If the alleles present at a given locus on a chromosome differ, that plant is heterozygous at that locus. A favorable allele of a marker is the allele of the marker that co-segregates with a desired phenotype (e.g., disease resistance). As used herein, when the favorable allele co-segregates with disease resistance, it is referred to as a “resistance allele.” As used herein, a QTL marker has a minimum of one favorable allele, although it is possible that the marker might have two or more favorable alleles found in the population. Any favorable allele of that marker can be used advantageously for the identification and construction of disease tolerant plant lines. Optionally, one, two, three or more favorable allele(s) of different markers are identified in, or introgressed into a plant, and can be selected for or against during MAS. Desirably, plants or germplasm are identified that have at least one such favorable allele that positively correlates with disease tolerance or improved disease tolerance. Alternatively, a marker allele that co-segregates with disease susceptibility also finds use with the invention, since that allele can be used to identify and counter select disease susceptible plants. Such an allele can be used for exclusionary purposes during breeding to identify alleles that negatively correlate with tolerance, to eliminate susceptible plants or germplasm from subsequent rounds of breeding.

As used herein, the term “bulk” refers to a method of managing a segregating population during inbreeding that involves growing the population in a bulk plot, harvesting the self pollinated seed of plants in bulk, and using a sample of the bulk to plant the next generation.

As used herein, the term “chromosome interval” designates a contiguous linear span of genomic DNA that resides in planta on a single chromosome. The term also designates any and all genomic intervals defined by any of the markers set forth in this invention. The genetic elements located on a single chromosome interval are physically linked and the size of a chromosome interval is not particularly limited. In some aspects, the genetic elements located within a single chromosome interval are genetically linked, typically with a genetic recombination distance of, for example, less than or equal to 20 cM, or alternatively, less than or equal to 10 cM. That is, two genetic elements within a single chromosome interval undergo meiotic recombination at a frequency of less than or equal to 20% or 10%, respectively. The boundaries of a chromosome interval can be defined by genetic recombination distance or by markers. In one embodiment, the boundaries of a chromosome interval comprise markers. In another embodiment, the boundaries of a chromosome interval comprise markers that will be linked to the gene controlling the trait of interest, i.e., any marker that lies within a given interval, including the terminal markers that defining the boundaries of the interval, and that can be used as a marker for the presents or absence of disease tolerance. In one embodiment, the intervals described herein encompass marker clusters that co-segregate with disease tolerance. The clustering of markers occurs in relatively small domains on the chromosomes, indicating the presence of a genetic locus controlling the trait of interest in those chromosome regions. The interval encompasses markers that map within the interval as well as the markers that define the terminal. An interval described by the terminal markers that define the endpoints of the interval will include the terminal markers and any marker localizing within that chromosome domain, whether those markers are currently known or unknown. Although it is anticipated that one skilled in the art may describe additional polymorphic sites at marker loci in and around the markers identified herein, any marker within the chromosome intervals described herein that are associated with disease tolerance fall within the scope of this claimed invention.

As used herein, “elite line” means any line that has resulted from breeding and selection for superior agronomic performance. Numerous elite lines are available and known to those of skill in the art of soybean breeding. An “elite population” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from and/or capable of giving rise to a plant with superior agronomic performance, such as an existing or newly developed elite line of soybean. In contrast, an “exotic line” or “exotic germplasm” is a line or germplasm derived from a plant not belonging to an available elite line or strain of germplasm. In the context of a cross between two plants or lines of germplasm, an exotic germplasm is not closely related by descent to the elite germplasm with which it is crossed. Most commonly, the exotic germplasm is not derived from any known elite line of a crop, but rather is selected to introduce genetic elements (typically desired alleles) into a breeding program.

As used herein, the term “denoting,” when used in reference to a plant genotype, refers to any method whereby a plant is indicated to have a certain genotype. Such indications of a certain genotype include, but are not limited to, any method where a plant is physically marked or tagged. Physical markings or tags that can be used include, but are not limited to, a barcode, a radio-frequency identification (RFID), a label or the like. Indications of a certain genotype also include, but are not limited to, any entry into any type of written or electronic database whereby the plant's genotype is provided.

As used herein, the term “locus” (or “loci”) refers to a chromosome region where a polymorphic nucleic acid, trait determinant, gene or marker is located. The loci of this invention comprise one or more polymorphisms in a population; i.e., alternative alleles are present in some individuals. A “gene locus” is a specific chromosome location in the genome of a species where a specific gene can be found.

As used herein, “linkage disequilibrium” or “LD” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Linked loci co-segregate more than 50% of the time, e.g., from about 51% to about 100% of the time. The term “physically linked” is sometimes used to indicate that two loci, e.g., two marker loci, are physically present on the same chromosome. Advantageously, the two linked loci are located in close proximity such that recombination between homologous chromosome pairs does not occur between the two loci during meiosis with high frequency, e.g., such that linked loci cosegregate at least about 90% of the time, e.g., 91%, 92%, 93%, 94%, 95%, 96%, 97%, 98%, 99%, 99.5%, 99.75%, or more of the time.

As used herein, “linkage group B2” corresponds to the soybean linkage group B2 described in Choi, et al., Genetics. 2007 May; 176(1): 685-696. Linkage group B2, as used herein, also corresponds to soybean chromosome 14 (as described on the World Wide Web at soybase.org/LG2Xsome.php).

As used herein, “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells that can be cultured into a whole plant.

As used herein, “genotype” means the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome interval.

As used herein, the term “introgressed”, when used in reference to a genetic locus, refers to a genetic locus that has been introduced into a new genetic background. Introgression of a genetic locus can thus be achieved through both plant breeding methods or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion. In certain embodiments, introgression could thus be achieved by substitution of a stem canker susceptibility locus with a corresponding stem canker resistance locus or by conversion of a locus from a stem canker susceptible genotype to a stem canker resistance genotype.

As used herein, the phrase “isolated nucleic acid molecule,” be it a naturally-occurring molecule or otherwise, refers to a nucleic acid molecule where the covalent bonds between that nucleic acid and other native nucleic acids that adjoin the isolated nucleic acid in its naturally occurring state have been broken or have been replaced with covalent bonds to non-native nucleic acids. An isolated nucleic acid molecule can be the predominant species present in a preparation. In certain embodiments, an isolated nucleic acid molecule can also be at least about 60% free, at least about 75% free, at least about 90% free, and at least about 95% free from other molecules (exclusive of solvent). The phrase “isolated nucleic acid molecule” thus does not encompass nucleic acid molecules present in their native chromosomal locations.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB. Ab, aB and ab. The null expectation is that there will be independent equal segregation into each of the four possible genotypes, i.e. with no linkage ¼ of the gametes will be of each genotype. Segregation of gametes into a genotypes differing from ¼ are attributed to linkage.

As used herein, the term “linked”, when used in the context of markers and/or genomic regions, means that the markers and/or genomic regions are located on the same linkage group or chromosome. The more tightly linked a marker is with a DNA locus influencing a phenotype, the more reliable the marker is for marker-assisted breeding, as the likelihood of a recombination event unlinking the marker and the locus decreases. Markers containing the causal mutation for a trait, or that are within the coding sequence of a causative gene, are ideal as no recombination is expected between them and the sequence of DNA responsible for the phenotype.

As used herein, “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus.

As used herein, “marker” also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

As used herein, “marker assay” means a method for detecting a polymorphism at a particular locus using a particular method. Marker assays thus include, but are not limited to, measurement of at least one phenotype (such as seed color, flower color, or other visually detectable trait as well as any biochemical trait), restriction fragment length polymorphism (RFLP), single base extension, electrophoresis, sequence alignment, allelic specific oligonucleotide hybridization (ASO), random amplified polymorphic DNA (RAPD), microarray-based polymorphism detection technologies, and the like.

As used herein, “phenotype” or “phenotypic trait” or “trait” refers to one or more trait of an organism. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is the result of several genes.

As used herein, “plant” refers to a whole plant any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

As used herein, “polymorphism” refers to the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, a “population of plants” or “plant population” refers to a set comprising any number, including one, of individuals, objects, or data from which samples are taken for evaluation, e.g. estimating QTL effects. Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silico representations of the plants. The population members need not be identical to the population members selected for use in subsequent cycles of analyses or those ultimately selected to obtain final progeny plants. Often, a plant population is derived from a single biparental cross, but may also derive from two or more crosses between the same or different parents. Although a population of plants may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

As used herein, “quantitative trait locus (QTL)” or “quantitative trait loci” (QTL) refers to a genetic domain that effects a phenotype that can be described in quantitative terms and can be assigned a “phenotypic value” which corresponds to a quantitative value for the phenotypic trait. A QTL can act through a single gene mechanism or by a polygenic mechanism. In some aspects, the invention provides QTL chromosome intervals, where a QTL (or multiple QTLs) that segregates with disease tolerance is contained in those intervals. As used herein, when a QTL (or multiple QTL) segregates with disease tolerance, it is referred to herein as a “resistance locus” (or “resistance loci”). In one embodiment of this invention, the boundaries of chromosome intervals are drawn to encompass markers that will be linked to one or more QTL. In other words, the chromosome interval is drawn such that any marker that lies within that interval (including the terminal markers that define the boundaries of the interval) is genetically linked to the QTL. Each interval comprises at least one QTL, and furthermore, may indeed comprise more than one QTL. Close proximity of multiple QTL in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifying the same QTL or two different QTL. Regardless, knowledge of how many QTL are in a particular interval is not necessary to make or practice the invention.

As used herein, the term “soybean” means Glycine max and includes all plant varieties that can be bred with soybean, including wild soybean species. In certain embodiments, soybean plants from the species Glycine max and the subspecies Glycine max L. ssp. max or Glycine max ssp. formosana can be genotyped using the compositions and methods of the present invention. In an additional aspect, the soybean plant is from the species Glycine soja, otherwise known as wild soybean, can be genotyped using these compositions and methods. Alternatively, soybean germplasm derived from any of Glycine max, Glycine max L. ssp. max, Glycine max ssp. Formosana, and/or Glycine soja can be genotyped using compositions and methods provided herein.

As used herein, the term “single nucleotide polymorphism (SNP)” means a polymorphism at a single site wherein the polymorphism constitutes any or all of a single base pair change, an insertion of one or more base pairs, and/or a deletion of one or more base pairs.

As used herein, the phrase “soybean stem canker” refers to any stem canker that is found on a soybean plant. Stem cankers found on soybean include, but are not limited to, those caused by Diaporthe phaseolorum var. meridionalis (DPM) and Diaporthe phaseolorum var. caulivora.

As used herein, the phrase “soybean stem canker resistance” refers to any form of resistance to a stem canker that can infect a soybean plant.

As used herein, the term “tolerance” or “improved tolerance” in a plant to disease conditions is an indication that the plant is less affected by disease conditions with respect to yield, survivability and/or other relevant agronomic measures, compared to a less tolerant, more “susceptible” plant. Tolerance is a relative term, indicating that a “tolerant” plant survives and/or produces better yields in disease conditions compared to a different (less tolerant) plant (e.g., a different soybean line strain) grown in similar disease conditions. As used in the art, disease “tolerance” is sometimes used interchangeably with disease “resistance.” One of skill will appreciate that plant tolerance to disease conditions varies widely, and can represent a spectrum of more-tolerant or less-tolerant phenotypes. However, by simple observation, one of skill can generally determine the relative tolerance or susceptibility of different plants, plant lines or plant families under disease conditions, and furthermore, will also recognize the phenotypic gradations of “tolerant.” When used in the context of stem canker resistance, “resistance” or “tolerance” refers to the ability of a soybean plant to exhibit a reduction in deleterious effects caused by stem canker infection.

As used herein, the term “yield” refers to the culmination of all agronomic traits as determined by the productivity per unit area of a particular plant product of commercial value. “Agronomic traits,” include the underlying genetic elements of a given plant variety that contribute to yield over the course of growing season.

### II. A CHROMOSOME INTERVAL ASSOCIATED WITH A STEM CANKER RESISTANCE PHENOTYPE

Provided herewith is a soybean chromosomal interval that is shown herein to be associated with a desirable stem canker resistance phenotype when present in certain allelic forms. The soybean chromosome interval is located on the telomere proximal end of the short arm of soybean linkage group B2 (chromosome 14).

In one embodiment, the present invention provides a plant comprising a nucleic acid molecule selected from the group consisting of SEQ ID NO: 1-77 fragments thereof, and complements of both. In another embodiment, the present invention also provides a plant comprising the alleles of the stem canker resistance locus, or fragments and complements thereof, as well as any plant comprising any combination of one or more disease resistance loci linked to at least one marker selected from the group consisting of SEQ ID NOs: 1-77. Such alleles may be homozygous or heterozygous.

The location in the soybean genome of the stem canker resistance locus and the chromosome interval comprising markers closely linked to it are disclosed in Table 1. Genetic map loci are represented in cM, with position zero being the first (most distal) marker known at the beginning of the chromosome on both Monsanto's internal consensus map and the GmConsensus 4.0 soybean genomic map, which is freely available to the public from the Soybase.org website and commonly used by those skilled in the art. Also disclosed in Table 1 are the physical locations of loci as they are reported on the Glyma1.0 public assembly by the US Department for Energy Joint Genome Institute (DOE-JGI) Community Sequencing Program (CSP), available on the phytozome.net website (Schmutz J, et al. (2010). “Genome sequence of the palaeopolyploid soybean.” Nature 463, 178-183).

TABLE 1
Genetic and physical map positions of markers in the chromosome
interval associated with stem canker resistance

| | Relative Genetic | | Physical Map | |
|---|---|---|---|---|
| | Map Position | | Position | |
| | MON | GmConsensus | (Glyma1.0) | |
| | Map | 4.0 B2 Map | Chr | |
| Marker/Locus Name | cM† | cM† | Start | Chr End |
| Glyma14g00200 | 5 | ** | 1587 | 2586 |
| Glyma14g00220 | 5 | ** | 7919 | 8092 |
| Glyma14g00500 | 6 | ** | 218397 | 219946 |
| Glyma14g00760 | 7 | ** | 383705 | 387995 |
| Glyma14g01010 | 8 | ** | 528766 | 532285 |
| Glyma14g01230 | 9 | ** | 660635 | 663095 |
| Glyma14g01420 | 10.1 | ** | 800723 | 803769 |
| Glyma14g01580 | 11 | ** | 912829 | 915637 |
| Glyma14g02010 | 11.9 | ** | 1181430 | 1184687 |
| SEQ ID NO. 1 | 11.9 | ** | 1185335 | 1185636 |
| SEQ ID NO. 2 | 12.1 | ** | 1212091 | 1212392 |
| SEQ ID NO. 3 | 12.2 | ** | 1215687 | 1215988 |
| SEQ ID NO. 4 | 12.3 | ** | 1241523 | 1241824 |
| Glyma14g02090 | 12.3 | ** | 1244773 | 1250879 |
| SEQ ID NO. 5 | 12.5 | ** | 1288220 | 1288521 |
| SEQ ID NO. 6 | 12.6 | ** | 1303361 | 1303662 |
| SEQ ID NO. 7 | 12.6 | ** | 1305710 | 1306501 |
| SEQ ID NO. 8 | 12.7 | ** | 1317537 | 1317838 |
| SEQ ID NO. 9 | 12.7 | ** | 1319525 | 1319826 |
| Glyma14g02190 | 12.7 | ** | 1328345 | 1328919 |
| SEQ ID NO. 10 | 12.8 | ** | 1340317 | 1340618 |
| SEQ ID NO. 11 | 12.9 | ** | 1359825 | 1359291 |
| SEQ ID NO. 12 | 12.9 | ** | 1360058 | 1360359 |
| Glyma14g02250 | 12.9 | ** | 1366447 | 1367151 |
| SEQ ID NO. 13 | 13.4 | ** | 1400262 | 1400563 |
| Glyma14g02330 | 13.4 | ** | 1402394 | 1402653 |
| Glyma14g02340 | 13.5 | ** | 1405226 | 1408718 |
| SEQ ID NO. 14 | 13.5 | ** | 1412188 | 1412489 |
| SEQ ID NO. 15 | 13.6 | ** | 1422513 | 1422814 |
| SEQ ID NO. 16 | 13.8 | ** | 1439549 | 1439850 |
| SEQ ID NO. 17 | 14 | ** | 1454634 | 1454935 |
| Glyma14g02400 | 14 | ** | 1455362 | 1457114 |
| SEQ ID NO. 18 | 14.3 | ** | 1482355 | 1482656 |
| SEQ ID NO. 19 | 14.4 | ** | 1487607 | 1487908 |
| Glyma14g02460 | 14.6 | ** | 1496360 | 1509265 |
| SEQ ID NO. 20 | 14.5 | ** | 1498778 | 1499079 |
| SEQ ID NO. 21 | 14.6 | ** | 1507759 | 1508060 |
| SEQ ID NO. 22 | 14.7 | ** | 1516669 | 1516970 |
| SEQ ID NO. 23 | 14.9 | ** | 1533736 | 1534037 |
| SEQ ID NO. 24 | 15 | ** | 1543479 | 1543780 |
| Glyma14g02500 | 15 | ** | 1546438 | 1549285 |
| SEQ ID NO. 25 | 15.3 | ** | 1560956 | 1561257 |
| Glyma14g02570 | 15.6 | ** | 1592525 | 1596286 |
| SEQ ID NO. 26 | 15.6 | ** | 1594309 | 1594610 |
| SEQ ID NO. 27 | 15.7 | ** | 1603504 | 1603805 |
| Glyma14g02620 | 16 | ** | 1621855 | 1625652 |
| SEQ ID NO. 28 | 16.2 | ** | 1645712 | 1646013 |
| SEQ ID NO. 29 | 16.5 | ** | 1666390 | 1666691 |
| Glyma14g02680 | 16.5 | ** | 1667934 | 1672212 |
| SEQ ID NO. 30 | 16.8 | ** | 1692422 | 1692723 |
| BARCSOYSSR_14_0091 | 17 | ** | 1711473 | 1711512 |
| SEQ ID NO. 31 | 17 | ** | 1714124 | 1714425 |
| Glyma14g02720 | 17.1 | ** | 1717374 | 1721919 |
| BARCSOYSSR_14_0092 | 17 | ** | 1717572 | 1717591 |
| SEQ ID NO. 32 | 17.1 | ** | 1718793 | 1719094 |
| SEQ ID NO. 33 | 17.1 | ** | 1719049 | 1719836 |
| SEQ ID NO. 34 | 17.1 | ** | 1719059 | 1719751 |
| Glyma14g02740 | 17.2 | ** | 1726998 | 1732095 |
| SEQ ID NO. 35 | 17.2 | ** | 1730281 | 1730582 |
| SEQ ID NO. 36 | 17.3 | ** | 1737792 | 1738093 |
| Glyma14g02770 | 17.5 | ** | 1750024 | 1752838 |
| SEQ ID NO. 37 | 17.6 | ** | 1756355 | 1756656 |
| SEQ ID NO. 38 | 17.9 | ** | 1784545 | 1784846 |
| Glyma14g02810 | 18 | ** | 1786228 | 1788914 |
| SEQ ID NO. 40 | 18 | ** | 1788509 | 1788837 |
| SEQ ID NO. 39 | 18 | ** | 1788654 | 1788955 |
| SEQ ID NO. 41 | 18.1 | ** | 1801585 | 1801886 |
| SEQ ID NO. 42 | 18.4 | ** | 1828285 | 1828754 |
| SEQ ID NO. 43 | 18.4 | ** | 1830218 | 1830519 |
| SEQ ID NO. 44 | 18.5 | ** | 1840314 | 1840615 |
| Sat_264 | ** | 10.953 | 1843791 | 1843842 |
| Glyma14g02900 | 18.6 | ** | 1844876 | 1846711 |
| SEQ ID NO. 45 | 18.6 | ** | 1846795 | 1847096 |
| Glyma14g02910 | 18.6 | ** | 1849715 | 1849950 |
| SEQ ID NO. 46 | 18.7 | ** | 1851467 | 1851768 |
| SEQ ID NO. 47 | 18.8 | ** | 1859587 | 1859888 |
| SEQ ID NO. 48 | 18.9 | ** | 1863986 | 1864287 |
| Glyma14g02980 | 19 | ** | 1881557 | 1886455 |
| SEQ ID NO. 49 | 19.2 | ** | 1888310 | 1888611 |
| SEQ ID NO. 50 | 19.3 | ** | 1896388 | 1896689 |
| SEQ ID NO. 51 | 19.3 | ** | 1899248 | 1899998 |
| SEQ ID NO. 52 | 19.4 | ** | 1907528 | 1907036 |
| SEQ ID NO. 53 | 19.4 | ** | 1910968 | 1911269 |
| Glyma14g03030 | 19.5 | ** | 1913942 | 1917435 |
| SEQ ID NO. 54 | 19.5 | ** | 1917382 | 1917683 |
| SEQ ID NO. 55 | 19.6 | ** | 1924658 | 1924959 |
| SEQ ID NO. 56 | 19.7 | ** | 1936381 | 1936682 |
| SEQ ID NO. 57 | 19.8 | ** | 1944506 | 1944807 |
| BARC-060237-16491 | 20 | ** | 1956678 | 1956981 |
| SEQ ID NO. 58 | 20.2 | ** | 1971009 | 1971310 |
| SEQ ID NO. 59 | 20.3 | ** | 1982756 | 1983057 |
| BARCSOYSSR_14_0107 | 20.5 | ** | 1998555 | 1998610 |
| SEQ ID NO. 60 | 20.6 | ** | 2006929 | 2007230 |
| SEQ ID NO. 61 | 20.7 | ** | 2014585 | 2014886 |
| SEQ ID NO. 62 | 20.7 | ** | 2017677 | 2017003 |
| SEQ ID NO. 64 | 20.9 | ** | 2034145 | 2034446 |
| SEQ ID NO. 63 | 20.9 | ** | 2035052 | 2036414 |
| Glyma14g03190 | 21 | ** | 2035930 | 2041929 |
| SEQ ID NO. 65 | 21.2 | ** | 2063523 | 2063824 |
| SEQ ID NO. 66 | 21.4 | ** | 2081414 | 2081715 |
| SEQ ID NO. 67 | 21.5 | ** | 2090831 | 2091132 |
| Glyma14g03270 | 21.5 | ** | 2091767 | 2093785 |
| SEQ ID NO. 68 | 21.6 | ** | 2096010 | 2096311 |
| SEQ ID NO. 69 | 21.7 | ** | 2107594 | 2107895 |
| SEQ ID NO. 70 | 21.8 | ** | 2110939 | 2111240 |
| SEQ ID NO. 71 | 21.9 | ** | 2115446 | 2115747 |
| SEQ ID NO. 72 | 22 | ** | 2122274 | 2122575 |
| Glyma14g03300 | 22 | ** | 2126211 | 2132189 |
| SEQ ID NO. 73 | 22.1 | ** | 2131085 | 2131386 |
| SEQ ID NO. 74 | 22.2 | ** | 2135082 | 2135383 |
| SEQ ID NO. 75 | 22.4 | ** | 2151701 | 2152002 |
| Glyma14g03350 | 22.5 | ** | 2158477 | 2160573 |
| Glyma14g03370 | 22.6 | ** | 2164798 | 2166953 |
| SEQ ID NO. 76 | 22.6 | ** | ** | ** |
| SEQ ID NO. 77 | 22.6 | ** | 2166492 | 2166793 |
| Glyma14g03380 | 22.6 | ** | 2168440 | 2170276 |
| BARCSOYSSR_14_0112 | 23 | ** | 2195518 | 2195569 |
| Glyma14g03410 | 23.1 | ** | 2203776 | 2206058 |
| Glyma14g03750 | 24 | ** | 2415322 | 2420285 |
| BARCSOYSSR_14_0130 | 25 | ** | 2525384 | 2525409 |
| Glyma14g03960 | 26 | ** | 2628626 | 2640732 |
| Glyma14g04110 | 27 | ** | 2738301 | 2742124 |
| Sat_342 | 28 | 15.5 | 2954822 | 2954895 |
| Glyma14g04740 | 29 | ** | 3269402 | 3277156 |
| BARCSOYSSR_14_0174 | 30 | ** | 3503969 | 3503988 |
| Glyma14g05330 | 31 | ** | 3772421 | 3774923 |
| Glyma14g05460 | 32 | ** | 3908497 | 3913092 |
| Glyma14g05640 | 33 | ** | 4067721 | 4069134 |

†cM = centiMorgans
**Exact coordinates not known. Coordinates can be estimated based on the nearest flanking loci with known coordinates.

In Table 1, “cM” refers to the classical definition of a centimorgan (Haldane 1919 J Genet 8:299-309) wherein one cM is equal to a 1% chance that a trait at one genetic locus will be separated from a trait at another locus due to crossing over in a single meiosis (meaning the traits cosegregate 99% of the time), and this definition is used herein to delineate map locations pertaining to this invention.

For example, the stem canker resistance chromosome interval contains SEQ ID NOs. 1-77 and is flanked by the markers Glyma14g00220 and Glyma14g05460, which are separated by approximately 27 cM on the internally-derived genetic map. This chromosome interval encompasses a marker cluster that co-segregates with stem canker tolerance in the populations studied at a −Log 10(P value)≧3.0. An example of a subinterval of the stem canker resistance chromosome interval is that which is flanked by SEQ ID NO. 1 and SEQ ID NO. 77, separated by approximately 10.7 cM on the internally-derived genetic map, that define a chromosome interval encompassing a cluster of markers that co-segregate with stem canker tolerance in the populations studied at a −Log 10(P value)≧3.0.

Thus, one skilled in the art can use this invention to improve the efficiency of breeding for improved disease tolerance in soybean by associating disease tolerance phenotypes with genotypes at previously unknown disease tolerance loci in the soybean genome. Disclosed herein are chromosome intervals that comprise alleles responsible for phenotypic differences between disease tolerant and disease susceptible soybean lines. Each chromosome interval is characterized by the genomic regions including and flanked by and including the markers Glyma14g00220 and Glyma14g05460 on chromosome B2, and comprise markers within or closely linked to (within 10 cM of) the stem canker resistance locus. This invention also comprises other intervals whose borders fall between, and including, those of Glyma14g00220 and Glyma14g05460, or any interval closely linked to those intervals.

Examples of markers useful for this purpose comprise the SNP markers listed in Table 1, or any marker that maps within the chromosome intervals described herein (including the termini of the intervals), or any marker linked to those markers. Such markers can be assayed simultaneously or sequentially in a single sample or population of samples.

Accordingly, the markers and methods of the present invention can be utilized to guide MAS or breeding soybean varieties with the desired complement (set) of allelic forms of chromosome intervals associated with superior agronomic performance (tolerance, along with any other available markers for yield, disease resistance, etc.). Any of the disclosed marker alleles can be introduced into a soybean line via introgression, by traditional breeding (or introduced via transformation, or both) to yield a soybean plant with superior agronomic performance. The number of alleles associated with tolerance that can be introduced or be present in a soybean plant of the present invention ranges from one to the number of alleles disclosed herein, each integer of which is incorporated herein as if explicitly recited.

Marker-assisted selection (MAS) using additional markers flanking either side of the DNA locus provide further efficiency because an unlikely double recombination event would be needed to simultaneously break linkage between the locus and both markers. Moreover, using markers tightly flanking a locus, one skilled in the art of MAS can reduce linkage drag by more accurately selecting individuals that have less of the potentially deleterious donor parent DNA. Any marker linked to or among the chromosome intervals described herein could be useful and within the scope of this invention.

Similarly, by identifying plants lacking the desired marker locus, susceptible or less tolerant plants can be identified, and, e.g., eliminated from subsequent crosses. Similarly, these marker loci can be introgressed into any desired genomic background, germplasm, plant, line, variety, etc., as part of an overall MAS breeding program designed to enhance yield. The invention also provides chromosome QTL intervals that find equal use in MAS to select plants that demonstrate disease tolerance or improved tolerance. Similarly, the QTL intervals can also be used to counter-select plants that are susceptible or have reduced tolerance to disease.

The present invention also extends to a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for alleles associated with tolerance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant of the present invention in that it comprises at least one of the allelic forms of the markers of the present invention, such that the progeny are capable of inheriting the allele.

Often, a method of the present invention is applied to at least one related soybean plant such as from progenitor or descendant lines in the subject soybean plants' pedigree such that inheritance of the desired tolerance allele can be traced. The number of generations separating the soybean plants being subject to the methods of the present invention will generally be from 1 to 20, commonly 1 to 5, and typically 1, 2, or 3 generations of separation, and quite often a direct descendant or parent of the soybean plant will be subject to the method (i.e., one generation of separation).

Thus, with this invention, one skilled in the art can detect the presence or absence of disease tolerance genotypes in the genomes of soybean plants as part of a marker assisted selection program. In one embodiment, a breeder ascertains the genotype at one or more markers for a disease tolerant parent, which contains a disease tolerance allele, and the genotype at one or more markers for a susceptible parent, which lacks the tolerance allele. For example, the markers of the present invention can be used in MAS in crosses involving elite ×exotic soybean lines by subjecting the segregating progeny to MAS to maintain disease tolerance alleles, or alleles associated with yield under disease conditions. A breeder can then reliably track the inheritance of the tolerance alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the disease tolerant parent can be reliably predicted to express the tolerant phenotype; progeny that share genotypes with the disease susceptible parent can be reliably predicted to express the susceptible phenotype. Thus, the laborious and inefficient process of manually phenotyping the progeny for disease resistance is avoided.

By providing the positions in the soybean genome of the intervals and the disease tolerance associated markers within, this invention also allows one skilled in the art to identify other markers within the intervals disclosed herein or linked to the chromosome intervals disclosed herein.

Closely linked markers flanking the locus of interest that have alleles in linkage disequilibrium with a resistance allele at that locus may be effectively used to select for progeny plants with enhanced tolerance to disease conditions. Thus, the markers described herein, such as those listed in Table 1, as well as other markers genetically or physically mapped to the same chromosome interval, may be used to select for soybean plants with enhanced tolerance to disease conditions. Typically, a set of these markers will be used, (e.g., 2 or more, 3 or more, 4 or more, 5 or more) in the flanking region above the gene and a similar set in the flanking region below the gene. Optionally, as described above, a marker within the actual gene and/or locus may also be used. The parents and their progeny are screened for these sets of markers, and the markers that are polymorphic between the two parents are used for selection. In an introgression program, this allows for selection of the gene or locus genotype at the more proximal polymorphic markers and selection for the recurrent parent genotype at the more distal polymorphic markers.

The choice of markers actually used to practice this invention is not particularly limited and can be any marker that maps within the stem canker resistance chromosome intervals described herein, any marker closely linked (within 10 cM) to a marker in the stem canker chromosome interval, or any marker selected from SEQ ID NOs: 1-77, or the markers listed in Table 1. Furthermore, since there are many different types of marker detection assays known in the art, it is not intended that the type of marker detection assay (e.g. RAPDs, RFLPs, SNPs, AFLPs, etc.) used to practice this invention be limited in any way.

Additional genetic markers can be used either in conjunction with the markers provided in Table 1 or independently of the markers provided in Table 1 to practice the methods of the instant invention. Publicly available marker databases from which useful markers can be obtained include, but are not limited to, the soybase.org website on the internet (World Wide Web) that is administered by the United States Agricultural Research Service, the United States Department of Agriculture, and Iowa State University. Additional soybean markers that can be used and that have been described in the literature include, but are not limited to, Hyten et al., BMC Genomics. 11:38, 2010; Choi et al., Genetics. 176(1):685-96, 2007; Yoon et al., Theor Appl Genet. 2007 March; 114(5):885-99; and Hyten et al. Crop Sci. 2010 50: 960-968.

Sequences for SEQ ID NO. 1-77 in Table 1 can be obtained from the Sequence Listing. Sequences for the publically available markers disclosed in Table 1 can be obtained on the World Wide Web (or Internet) using the identifiers provided in Column 1 (Marker/Locus Name) from the following internet locations:

a) “soybase.org” (described in Grant et al., Nucleic Acids Research, 2010, Vol. 38, Database issue D843-D846) or soybase.org/gbrowse/cgi-bin/gbrowse/gmax1.01/(see Hyten D L, Choi I-Y, Song Q, Specht J E, Carter T E et al. (2010) A high density integrated genetic linkage map of soybean and the development of a 1,536 Universal Soy Linkage Panel for QTL mapping. Crop Science 50:960-968 and Hyten D L, Cannon S B, Song Q, Weeks N, Fickus E W et al. (2010). High-throughput SNP discovery through deep resequencing of a reduced representation library to anchor and orient scaffolds in the soybean whole genome sequence. BMC Genomics 11(1): 38).

b) “phytozome.net” or “phytozome.net/cgi-bin/gbrowse/soybean/”;

c) “www.plantgdb.org” or “plantgdb.org/GmGDB/(Assembly version Glyma1.170 (April 2009)”; and,

d) “ncbi.nlm.nih.gov/sites/entrez” and subsites “ncbi.nlm.nih.gov/nucest”, “ncbi.nlm nih.gov/dbEST”, “ncbi.nlm nih.gov/genbank/”, “.ncbi.nlm nih.gov/sites/genome”, “ncbi.nlm nih.gov/unigene”, and “ncbi.nlm.nih.gov/UniGene/UGOrg.cgi?TAXID=3847”.

### III. IDENTIFICATION OF PLANTS EXHIBITING THE STEM CANKER RESISTANT PHENOTYPE

To observe the presence or absence of the stem canker resistance phenotypes, soybean plants comprising genotypes of interest can be exposed to stem cankers in seedling stages, early to mid-vegetative growth stages, or in early reproductive stages. The design and execution of stem canker exposure experiments to assess tolerance have been described in numerous publications including, but not limited to, Pioli et al. Phytopathology 93:136-146, 2330; and Keeling, Plant Disease 72:217-220, 1988. In certain embodiments, the hypocotyls of seedlings or the stems of plants can be inoculated with Diaporthe by insertion of toothpicks or other devices comprising the fungi. Resistance can be determined by exposing the plants to stem cankers and measuring any plant growth feature that is impacted by stem canker infestation. In certain embodiments, resistance can be assessed by measuring a soybean yield parameter. Soybean yield parameters that can be examined to assess stem canker tolerance include, but are not limited to, average seed weight, average seeds per pod, average number of pods per plant, chlorophyll content.

A rating scale that evaluates the degree of stem canker resistance can also be employed to identify “stem canker susceptible” and “stem canker resistance” plants. An exemplary and non limiting scale for evaluating the stem canker susceptibility phenotype is as follows, where the low numbers correspond to an “stem canker resistance” phenotype and the high numbers correlate to an “stem canker susceptible” phenotype.

An exemplary rating and damage system that can be used in stem inoculation or other assays is a Percentage of Dead Plants Rating is as described in Table 2.

TABLE 2
Description of an exemplary rating scale used for stem canker resistance
phenotyping

| Rating | Percentage of Dead Plants (% DP) |
|---|---|
| R = Resistant | 0-25% DP |
| MR = Moderately resistant | 26-50% DP |
| MS = Moderately susceptible | 51-75% DP |
| S = Susceptible | 76-90% DP |
| HS = Highly susceptible | Above 90% DP |

The percentage of dead plants can be calculated using the formula:

$$Percentage of Dead Plants or \% DP Score ( per pot ) \text{:} \frac{\# of Dead Plants + ( \# of Infected Plants / 2 )}{Total Number of Plants} \times 100$$

In certain embodiments, the plants can be assigned a damage index (DI), which is calculated using the following formula:

$$DI = \sum \frac{( Each scale \times Number of plants in the scale )}{4 \times Total number of plants evaluated} \times 100$$

In this formula, a higher damage index corresponds to a more susceptible plant.

In other embodiments, a 0-5 scale essentially similar to that described by Keeling in Plant Disease 72:217-220, 1988 can be used. In brief, stems are inoculated by insertion of a toothpick or other device, the plants are permitted to grow for a sufficient period of time for disease to be manifest (about 21 to about 24 days in typical growing conditions), the stems are split and the distance of disease progression from the site of inoculation to the base of the stem is measured. This distance is converted to a disease rating value as follows: 0=no lesions, 1=1 mm or less, 2=1.1 to 3 mm, 3=3.1 to 6 mm, 4=6.1 to 10 mm, 5=10 mm or more. Plants with the highest scores are the least resistant such that plants scoring a “5” have chlorotic stems and are dying.

### IV. INTROGRESSION OF A GENOMIC REGION ASSOCIATED WITH A STEM CANKER RESISTANCE PHENOTYPE

Also provided herewith are unique soybean germplasms comprising an introgressed genomic region that is associated with a stem canker resistance phenotype and methods of obtaining the same. Marker-assisted introgression involves the transfer of a chromosomal region, defined by one or more markers, from one germplasm to a second germplasm. Offspring of a cross that contain the introgressed genomic region can be identified by the combination of markers characteristic of the desired introgressed genomic region from a first germplasm (i.e. such as a stem canker resistance germplasm) and both linked and unlinked markers characteristic of the desired genetic background of a second germplasm (i.e. a stem canker susceptible germplasm). In addition to the markers provided herewith that identify alleles of genomic region that is associated with a stem canker resistance phenotype, flanking markers that fall on both the telomere proximal end of the genomic region on linkage group B2 (chromosome 14) and the centromere proximal end of the linkage group B2 (chromosome 14) genomic region are also provided in Table 1. Such flanking markers are useful in a variety of breeding efforts that include, but are not limited to, introgression of the genomic region associated with a stem canker resistance phenotype into a genetic background comprising markers associated with germplasm that ordinarily contains the allelic forms of the genomic region that is associated with a “stem canker susceptible” phenotype. Numerous markers that are linked and either immediately adjacent or adjacent to a linkage group B2 stem canker resistance QTL in soybean that permit introgression of the stem canker resistance QTL in the absence of extraneous linked DNA from the source germplasm containing the QTL are provided herewith. In certain embodiments, the linked and immediately adjacent markers are within about 105 kilobases (kB), 80 kB, 60 kB, 50 kB, 40 kB, 30 kB, 20 kB, 10 kB, 5 kB, 1 kB, 0.5 kB, 0.2 kB, or 0.1 kB of the introgressed genomic region. In certain embodiments, the linked and adjacent markers are within 1,000 kB, 600 kB, 500 kB, 400 kB, 300 kB, 200 kB, 150 kB of the introgressed genomic region. In certain embodiments, genomic regions comprising some or all of a stem canker resistance QTL on linkage group B2 (chromosome 14) that are delimited by the markers of Table 1 can be introgressed into the genomes of susceptible varieties by using markers that include, but are not limited to, adjacent markers and/or immediately adjacent markers provided in Table 1. Those skilled in the art will appreciate that when seeking to introgress a smaller genomic region comprising a stem canker resistance locus of Table 1 that any of the telomere proximal or centromere proximal markers that are immediately adjacent to a larger genomic region comprising a stem canker resistance locus can also be used to introgress that smaller genomic region.

Provided herein are methods of introgressing any of the genomic regions comprising a linkage group B2 stem canker resistance locus of Table 1 into soybean germplasm that lacks such a linkage group B2 stem canker resistance locus. In certain embodiments, the soybean germplasm that lacks such a genomic region comprising linkage group B2 stem canker resistance locus is stem canker susceptible or has less than optimal levels of stem canker resistance. In certain embodiments, the methods of introgression provided herein can yield soybean plants comprising introgressed genomic regions comprising a linkage group B2 stem canker resistance locus of Table 1 where the immediately adjacent genomic DNA and/or some or all of the adjacent genomic DNA between the introgressed genomic region and the telomere or centromere will comprise allelic forms of the markers of Table 1 that are characteristic of the germplasm into which the genomic region is introgressed and distinct from the germplasm from which the genomic region is derived. In certain embodiments, the soybean germplasm into which the genomic region is introgressed is germplasm that lacks such a linkage group B2 stem canker resistance locus. In certain embodiments, the soybean germplasm into which the genomic region is introgressed is germplasm that lacks such a linkage group B2 stem canker resistance locus and is either stem canker susceptible or has less than optimal levels of stem canker resistance. In certain embodiments, the germplasm from which the linkage group B2 stem canker resistance locus is obtained comprises Tracy-M or germplasm derived from Tracy-M.

Also provided herein are soybean plants produced by the aforementioned methods of introgression. In certain embodiments, such soybean plants will comprise introgressed genomic regions comprising a linkage group B2 stem canker resistance locus of Table 1 where the immediately adjacent genomic DNA and/or some or all of the adjacent genomic DNA between the introgressed genomic region and the telomere or centromere will comprise allelic forms of the markers of Table 1 that are characteristic of the germplasm into which the genomic region is introgressed and distinct from the germplasm from which the genomic region is derived. In an exemplary embodiment where a genomic region flanked by, or including, markers SEQ ID NO. 1 and SEQ ID NO. 77 is introgressed, plants comprising that linkage group B2 genomic region containing a stem canker resistance locus wherein one or more of the adjacent or immediately adjacent telomere proximal markers of Table 1 can comprise allelic forms that are characteristic of the germplasm into which the genomic region is introgressed and/or that are distinct from the germplasm from which the genomic region is derived.

Additional markers are located on linkage group B2 (chromosome 14) and other chromosomes and may be useful for introgressing a linkage group B2 soybean stem canker resistance QTL. Publicly available marker databases from which additional useful markers located on linkage group B2 (chromosome 14) and other chromosomes can be obtained include, but are not limited to, the soybase.org website on the internet that is administered by the United States Agricultural Research Service, the United States Department of Agriculture, and Iowa State University. Soybean plants or germplasm comprising an introgressed genomic region that is associated with a stem canker resistance phenotype wherein at least 10%, 25%, 50%, 75%, 90%, or 99% of the remaining genomic sequences carry markers characteristic of soybean plants or germplasm that are otherwise or ordinarily comprise a genomic region associated with the stem canker susceptible phenotype are thus provided. Furthermore soybean plants comprising an introgressed region where closely linked regions adjacent and/or immediately adjacent to the linkage group B2 regions provided herewith that comprise genomic sequences carrying markers characteristic of soybean plants or germplasm that are otherwise or ordinarily comprise a genomic region associated with the stem canker susceptible phenotype are also provided.

### V. SOYBEAN PLANTS COMPRISING A GENOMIC REGION ASSOCIATED WITH A STEM CANKER RESISTANCE PHENOTYPE

Also provided herein are soybean plants comprising linkage group B2 genomic regions associated with a stem canker resistance phenotype wherein immediately adjacent genomic regions and/or one or more adjacent genomic regions characteristic of soybean germplasms that lack the genomic regions associated with a stem canker resistance phenotype and/or that are distinct from the germplasm from which the genomic region is derived. In certain embodiments, such plants can be produced by the aforementioned methods of introgression. In certain embodiments, soybean plants comprising a linkage group B2 stem canker resistance locus of Table 1 where the immediately adjacent genomic DNA and/or some or all of the adjacent genomic DNA between the introgressed genomic region and the telomere or centromere will comprise allelic forms of the markers of Table 1 that are characteristic of germplasms that lack the linkage group B2 genomic regions of Table 1 comprising a stem canker resistance phenotype and/or that are distinct from the germplasm from which the genomic region is derived.

Also provided herein are soybean plants comprising genomic regions containing the stem canker resistance loci. In certain embodiments, such soybean plants will comprise introgressed genomic regions comprising a linkage group B2 stem canker resistance locus of Table 1 where the immediately adjacent genomic DNA and/or some or all of the adjacent genomic DNA between the introgressed genomic region and the telomere or centromere will comprise allelic forms of the markers of Table 1 that are characteristic of the germplasm into which the genomic region is introgressed and distinct from the germplasm from which the genomic region is derived. In an exemplary embodiment where a genomic region flanked by, or including, markers SEQ ID NO. 1 and SEQ ID NO. 77 is introgressed, plants comprising that linkage group B2 genomic region containing a stem canker resistance locus wherein one or more of the adjacent or immediately adjacent telomere proximal markers of Table 1 and one or more of the adjacent centromere or immediately adjacent centromere proximal markers of Tables 1 can comprise allelic forms that are characteristic of the germplasm into which the genomic region is introgressed and/or that are distinct from the germplasm from which the genomic region is derived.

As used herein, a maturity group refers to an industry division of groups of varieties based range in latitude which the plant is best adapted and most productive. Soybean varieties are classified into 13 recognized maturity groups with the designations ranging from maturity groups 000, 00, 0, and I through X, wherein 000 represents the earliest maturing variety and X represents the latest maturing variety. Soybean plants in maturity groups 000 to IV have indeterminate plant habit, while soybean plants in maturity groups V through X have determinate plant habit. Herein, determinate growth habit refers to a cease vegetative growth after the main stem terminates in a cluster of mature pods. Herein, indeterminate growth habit refers to the development of leaves and flowers simultaneously throughout a portion of their reproductive period, with one to three pods at the terminal apex. Early maturity varieties (000 to IV) are adapted to northern latitudes with longer day lengths with the maturity designation increasing in southern latitudes with shorter day lengths.

Herein, relative maturity refers to a soybean plant maturity group subdividing a maturity group into tenths, for example 111.5. Relative maturity provided a more exact maturity. The number following the decimal point refers to the relative earliness or lateness with a maturity group, examples of which including IV.2 is an early group IV variety and IV.9 is a late group IV.

It is further understood that a soybean plant of the present invention may exhibit the characteristics of any relative maturity group. In an aspect, the relative maturity group is selected from the group consisting of 000.1-000.9, 00.1-00.9, 0.1-0.9, I.1-I.9, II.1-II.9, III.1-III.9, IV.1-IV.9, V.1-V.9, VI.1-VI.9, VII.1-VII.9, VIII.1-VIII.9, IX.1-IX.9, and X.1-X.9. The pollen for selected soybean plant can be cryopreserved and used in crosses with soybean lines from other maturity groups to introgress a stem canker resistance locus in a line that would not normally be available for crossing in nature. Pollen cryopreservation techniques are well known in the art (Tyagi and Hymowitz, Cryo letters 24: 119-124 (2003), Liang et al. Acta Botanica Sinica 35: 733-738 (1993)).

### VI. SOYBEAN DONOR PLANTS COMPRISING GENOMIC REGION ASSOCIATED WITH THE STEM CANKER RESISTANCE PHENOTYPES

A stem canker resistant QTL allele or alleles can be introduced from any plant that contains that allele (donor) to any recipient soybean plant. In one aspect, the recipient soybean plant can contain additional stem canker resistant loci. In another aspect, the recipient soybean plant can contain a transgene. In another aspect, while maintaining the introduced QTL, the genetic contribution of the plant providing the stem canker resistant QTL can be reduced by back-crossing or other suitable approaches. In one aspect, the nuclear genetic material derived from the donor material in the soybean plant can be less than or about 50%, less than or about 25%, less than or about 13%, less than or about 5%, 3%, 2% or 1%, but that genetic material contains the stem canker resistant locus or loci of interest

Plants containing one or more stem canker resistant loci described can be donor plants. Stem canker plants containing resistant loci can be, examples of which including screened for by using a nucleic acid molecule capable of detecting a marker polymorphism associated with resistance. Soybean donor plants comprising a genomic region containing a linkage group B2 stem canker resistance locus include, but are not limited to, Tracy-M and derivatives thereof. In certain embodiments, a donor plant can be a susceptible line. In certain embodiments, a donor plant can also be a recipient soybean plant

Also provided herewith are additional soybean plants that comprising a genomic region associated with a stem canker resistance phenotype that are identified by use of the markers provided in Table 1 and/or methods provided herein. Any of the soybean plants identified herein or other soybean plants that are otherwise identified using the markers or methods provided herein can be used in methods that include, but are not limited to, methods of obtaining soybean plants with an introgressed stem canker resistance locus, obtaining a soybean plant that exhibits a stem canker resistance phenotype, or obtaining a soybean plant comprising in its genome a genetic region associated with a stem canker resistance phenotype.

In certain embodiments, the soybean plants provided herein or used in the methods provided herein can comprise a transgene that confers tolerance to glyphosate. Transgenes that can confer tolerance to glyphosate include, but are not limited to, transgenes that encode glyphosate tolerant Class I EPSPS (5-enolpyruvylshikimate-3-phosphate synthases) enzymes or glyphosate tolerant Class II EPSPS (5-enolpyruvylshikimate-3-phosphate synthases) enzymes. Useful glyphosate tolerant EPSPS enzymes provided herein are disclosed in U.S. Pat. No. 6,803,501, RE39,247, U.S. Pat. No. 6,225,114, U.S. Pat. No. 5,188,642, and U.S. Pat. No. 4,971,908. In certain embodiments, the glyphosate tolerant soybean plants can comprise a transgene encoding a glyphosate oxidoreductase or other enzyme which degrades glyphosate. Glyphosate oxidoreductase enzymes had been described in U.S. Pat. No. 5,776,760 and U.S. Reissue Pat. No. 38,825. In certain embodiments the soybean plant can comprise a transgene encoding a glyphosate N-acetyltransferase gene that confers tolerance to glyphosate. In certain embodiments, the soybean plant can comprise a glyphosate n-acetyltransferase encoding transgene such as those described in U.S. Pat. No. 7,666,644. In still other embodiments, soybean plants comprising combinations of transgenes that confer glyphosate tolerance are provided. Soybean plants comprising both a glyphosate resistant EPSPS and a glyphosate N-acetyltransferase are also provided herewith. In certain embodiments, it is contemplated that the soybean plants used herein can comprise one or more specific genomic insertion(s) of a glyphosate tolerant transgene including, but not limited to, as those found in: i) MON89788 soybean (deposited under ATCC accession number PTA-6708 and described in US Patent Application Publication Number 20100099859), ii) GTS 40-3-2 soybean (Padgette et al., Crop Sci. 35: 1451-1461, 1995), iii) event 3560.4.3.5 soybean (seed deposited under ATCC accession number PTA-8287 and described in US Patent Publication 20090036308), or any combination of i (MON89788 soybean), ii (GTS 40-3-2 soybean), and iii (event 3560.4.3.5 soybean).

An stem canker resistance QTL of the present invention may also be introduced into an soybean line comprising one or more transgenes that confer tolerance to herbicides including, but not limited to, glufosinate, dicamba, chlorsulfuron, and the like, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress resistant, increased digestibility, industrial enzymes, pharmaceutical proteins, peptides and small molecules, improved processing traits, improved flavor, nitrogen fixation, hybrid seed production, reduced allergenicity, biopolymers, and biofuels among others. These agronomic traits can be provided by the methods of plant biotechnology as transgenes in soybean.

In certain embodiments, it is contemplated that genotypic assays that provide for non-destructive identification of the plant or plants can be performed either in seed, the emergence stage, the “VC” stage (i.e. cotyledons unfolded), the V1 stage (appearance of first node and unifoliate leaves), the V2 stage (appearance of the first trifoliate leaf), and thereafter. In certain embodiments, non-destructive genotypic assays are performed in seed using apparati and associated methods as described in U.S. Pat. Nos. 6,959,617; 7,134,351; 7,454,989; 7,502,113; 7,591,101; 7,611,842; and 7,685,768, which are incorporated herein by reference in their entireties. In certain embodiments, non-destructive genotypic assays are performed in seed using apparati and associated methods as described in US Patent Application Publications 20100086963, 20090215060, and 20090025288, which are incorporated herein by reference in their entireties. Published U.S. Patent Applications US 2006/0042527, US 2006/0046264, US 2006/0048247, US 2006/0048248, US 2007/0204366, and US 2007/0207485, which are incorporated herein by reference in their entirety, also disclose apparatus and systems for the automated sampling of seeds as well as methods of sampling, testing and bulking seeds. Thus, in a certain embodiments, any of the methods provided herein can comprise screening for markers in individual seeds of a population wherein only seed with at least one genotype of interest is advanced.

### VII. MOLECULAR ASSISTED BREEDING TECHNIQUES

Genetic markers that can be used in the practice of the instant invention include, but are not limited to, are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties (U.S. Pat. No. 6,207,367). These markers can form a basis for determining associations with phenotype and can be used to drive genetic gain. The implementation of marker-assisted selection is dependent on the ability to detect underlying genetic differences between individuals.

Certain genetic markers for use in the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

In another embodiment, markers that include, but are not limited, to single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with stem canker resistance loci, regions flanking stem canker resistance loci, regions linked to stem canker resistance loci, and/or regions that are unlinked to stem canker resistance loci can be used in certain embodiments of the instant invention.

In one embodiment, nucleic acid-based analyses for determining the presence or absence of the genetic polymorphism (i.e. for genotyping) can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (Genotypes) that comprise or are linked to a genetic marker that is linked to or correlated with stem canker resistance loci, regions flanking stem canker resistance loci, regions linked to stem canker resistance loci, and/or regions that are unlinked to stem canker resistance loci can be used in certain embodiments of the instant invention.

Herein, nucleic acid analysis methods include, but are not limited to, PCR-based detection methods (for example, TaqMan assays), microarray methods, mass spectrometry-based methods and/or nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form.

Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. However, the compositions and methods of the present invention can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

Target nucleic acid sequence can also be detected by probe linking methods as disclosed in U.S. Pat. No. 5,616,464, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283. SBE methods are based on extension of a nucleotide primer that is adjacent to a polymorphism to incorporate a detectable nucleotide residue upon extension of the primer. In certain embodiments, the SBE method uses four synthetic oligonucleotides. Two of the oligonucleotides serve as PCR primers and are complementary to sequence of the locus of genomic DNA which flanks a region containing the polymorphism to be assayed. Following amplification of the region of the genome containing the polymorphism, the PCR product is mixed with the third and fourth oligonucleotides (called extension primers) which are designed to hybridize to the amplified DNA adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, the locus or loci of interest can be directly sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays, as reviewed by R. F. Service Science 2006 311:1544-1546.

The markers to be used in the methods of the present invention should preferably be diagnostic of origin in order for inferences to be made about subsequent populations. Experience to date suggests that SNP markers may be ideal for mapping because the likelihood that a particular SNP allele is derived from independent origins in the extant populations of a particular species is very low. As such, SNP markers appear to be useful for tracking and assisting introgression of QTLs, particularly in the case of genotypes.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Inoculation and Assessment of Tolerance Phenotypes

Soybean plants were inoculated 12-15 days after planting by inserting a toothpick infested with Diaporthe phaseolorum f. sp. meridionalis (DPM) through the stem, approximately 0.5-1″ below the cotyledon leaves (ends of toothpick should just protrude from the sides of the stem). Inoculate 15 plants per pot Immediately, after inoculating up to two pots, place pots back in the humid tent (created by covering the plants with plastic and turning on the misting system). High humidity conditions (>85%) after inoculation are necessary to accelerate the infection process. When inoculations are completed, mister is set for conditions to provide >85% relative humidity inside humid tent.

Soybean plants are rated between 21 and 24 days after inoculation, or when known susceptible checks visually show symptoms associated with susceptibility (dead, dying plants). The number of plants inoculated, the number of dead plants, and the number of infected, severely-infected, and resistant plants is determined by splitting the stems. Plants are assigned the following ratings based on visual symptoms: (1) Resistant: Plants that do not show any browning along the inside of the stem, only a small browning around the toothpick wound. (2) Infected: Plants that show browning inside the stem around the toothpick area that extends somewhat along the stem. (3) Severely-infected plants: Plants that show a solid browning inside the stem that covers most of the stem, the outside of the stem looks dried out around the toothpick wound and progresses along the stem, a sign that the plant will eventually die. A severely infected plant is considered a dead plant.

Plants were rated 21-24 days after inoculation by determining percent mortality in each F3 family Percent mortality was calculated using the aforementioned formula described in section III: Identification of Plants Exhibiting the Stem canker Resistant Phenotype. A disease rating was assigned based on the scale described in Table 2.

A trifoliolate leaflet was taken from each plant at inoculation, and all of the leaflets from each family were combined as a bulk for DNA extraction. The parents were fingerprinted with a panel of SNP markers to find DNA marker polymorphisms.

### Example 2

### Assays Useful for Detecting Stem Canker Tolerance Genotypes

For convenience, primer sequences for amplifying SNP marker loci linked to stem canker resistance loci B2 and the probes used to genotype the corresponding SNP sequences are provided in Table 3. In a few cases, primers and probes are not available because they were generated by one of several companies that provide such services to the public. Primer and probe synthesis is also within the skill of the art once the SNP position in the soybean genome is provided. One of skill in the art will also immediately recognize that other sequences to either side of the given primers can be used in place of the given primers, so long as the primers can amplify a region that includes the allele to be detected. Further, it will be appreciated that the precise probe to be used for detection can vary, e.g., any probe that can identify the region of a marker amplicon to be detected can be substituted for those examples provided herein. Also, configuration of the amplification primers and detection probes can, of course, vary. Thus, the invention is not limited to the primers, probes, or marker sequences specifically recited herein.

TABLE 3
Primers and probes useful for detecting Stem canker resistance

| Marker | | | | SEQ ID NO. | | | |
|---|---|---|---|---|---|---|---|
| SEQ | SNP | | | Forward | Reverse | Probe | |
| ID NO | Position | Allele 1 | Allele 2 | Primer | Primer | 1 | Probe 2 |
| 34 | 315 | T | C | 78 | 79 | 80 | 81 |
| 7 | 186 | GAGT | * | 82 | 83 | 84 | 85 |
| | | TCG | | | | | |
| 9 | 201 | T | C | 86 | 87 | 88 | 89 |
| 33 | 486 | AT | * | 90 | 91 | 92 | 93 |
| 42 | 371 | T | G | 94 | 95 | 96 | 97 |
| 51 | 220 | A | T | 98 | 99 | 100 | 101 |
| 52 | 361 | C | T | 102 | 103 | 104 | 105 |
| 62 | 366 | T | C | 106 | 107 | 108 | 109 |
| 63 | 1026 | A | T | 110 | 111 | 112 | 113 |

Illustrative stem canker resistance marker DNA sequences SEQ TD NOs: 34, 7, 9, 33, 42, 51, 52, 62, or 63 can be amplified using the primers indicated in Table 3 using the SEQ ID NOs in the “Forward Primer” and “Reverse Primer columns” and detected with the probes indicated in Table 3 using the SEQ ID NOs in the “Probe 1” and “Probe 2” columns.

### Example 3

### Marker Trait Association Studies

A mapping population was developed by crossing individual plants from Tracy-M (stem canker resistant) and AG4801 (stem canker susceptible) soybean lines. Plants from 100 F2:3 families were inoculated and phenotyped for stem canker resistance using the method described in Example 1. A trifoliolate leaflet was taken from each plant at inoculation, and all of the leaflets from each family were combined as a bulk for DNA extraction and genotyped with a panel of SNP markers that were selected to collectively span the soybean genome. Loci were eliminated from further analysis where they were monomorphic in the subject population studied. The parents were also fingerprinted with a panel of SNP markers to find DNA marker polymorphisms. The Tracy-M derived population was found to have a QTL on linkage group B2 and marker SEQ ID NO. 34 was most significantly associated with the stem canker resistance trait.

Historical phenotypic and genotypic data from a set of 1429 pre-commercial soybean varieties was analyzed. A marker-trait association study for the stem canker resistance trait was performed using association mapping of single-markers in GenABEL package in R and association mapping of haplotypes in glmnet package in R (Aulchenko et al., 2007, Bioinformatics. 23(10):1294-1296; Friedman et al., 2010, Journal of Statistical Software. 33(1):1-22; R project for statistical computing available at the r-project.org website).

Table 4 contains the results of an historic marker-trait association study. Several markers were found to be strongly associated with the stem canker resistance phenotype (threshold of −log 10(pvalue)≧3.0). These markers comprise the linkage group B2 stem canker resistance QTL.

TABLE 4
Results of an historic marker-trait association study for
stem canker resistance.

| Marker | Association with stem |
|---|---|
| SEQ ID | canker phenotype |
| NO. | (−log10(pvalue)) |
| 1 | 8.1 |
| 2 | 9.0 |
| 3 | 5.4 |
| 4 | 5.6 |
| 5 | 3.7 |
| 9 | 3.7 |
| 10 | 4.9 |
| 11 | 5.8 |
| 12 | 6.8 |
| 13 | 6.7 |
| 14 | 8.0 |
| 15 | 8.1 |
| 16 | 12.6 |
| 17 | 11.4 |
| 18 | 12.6 |
| 19 | 11.8 |
| 20 | 12.3 |
| 21 | 12.3 |
| 22 | 12.6 |
| 23 | 9.7 |
| 24 | 67.5 |
| 25 | 66.3 |
| 26 | 92.4 |
| 27 | 111.3 |
| 28 | 117.3 |
| 29 | 115.8 |
| 30 | 110.5 |
| 34 | 147.7 |
| 35 | 177.7 |
| 37 | 166.8 |
| 38 | 171.3 |
| 39 | 118.8 |
| 40 | 118.8 |
| 41 | 145.8 |
| 43 | 2.3 |
| 44 | 157.3 |
| 45 | 113.3 |
| 46 | 153.3 |
| 48 | 113.1 |
| 49 | 138.5 |
| 53 | 157.3 |
| 56 | 138.0 |
| 57 | 137.8 |
| 59 | 138.0 |
| 60 | 123.4 |
| 61 | 5.6 |
| 62 | 59.8 |
| 64 | 81.7 |
| 66 | 8.7 |
| 67 | 8.4 |
| 69 | 3.7 |
| 72 | 5.8 |
| 73 | 3.0 |
| 74 | 3.0 |
| 75 | 4.7 |
| 76 | 4.4 |
| 77 | 7.8 |

Thus, the marker-trait association analysis revealed that markers within the interval flanked by and including markers SEQ ID NO. 1 and SEQ ID NO. 77 were highly associated with stem canker resistance (−log 10(pvalue)≧3.0). Markers bordering the stem canker resistance locus also provide utility with this invention, but their associations with that interval tend to decrease as their locations become further removed from the stem canker resistance locus.

### Example 4

### Detecting Resistance in a Population of Plants and Monitoring the Introgression of Resistance Loci from One Plant Line into Another Via MAS

A population of soybean plants can be phenotyped using any method that gauges the effect of Stem canker infection on a plant trait, including the methods described herein. The genotypes of the plants in the population at one or more markers that map to the Stem canker resistanct locus chromosome interval, or at one or more markers closely linked to the interval, can also be determined. In one embodiment, statistical associations can then be made between the recorded phenotypes and the genotypes using a variety of methods known in the art, including those described herein.

In one embodiment, genotypes of offspring derived from one or more individuals in the population can be compared to the genotypes of the parents at one or more marker loci linked to the Stem canker locus genotypes of the parents at the same locus. Individuals that share marker genotypes with the resistant parent at one or more markers can then be selected for advancement in the breeding program. Individuals that do not share marker genotypes with the resistant parent, or individuals that do share marker genotypes with the susceptible parent, can be discarded. This process saves the laborious and time-consuming process of phenotyping plants to verify which are resistant or susceptible.

In some embodiments, useful markers comprise any marker that is within or genetically linked to the stem canker resistance locus. In other embodiments, useful markers comprise any marker that is between publically available markers Glyma14g00220 and Glyma14g05460.

Selections and assays may be performed on single loci, or simultaneously on multiple loci. For example, a breeder skilled in the art could base advancement decisions on the genotypes of markers linked to the stem canker resistance locus and genotypes of markers linked to other loci, simultaneously. For instance, a breeder may require that the same plant must exhibit genotypes at one or more markers linked to the stem canker resistance locus and/or at one or more markers linked to any other locus in order to be advanced. In one embodiment, a breeder may require that the same plant must exhibit genotypes at one or more markers linked to the stem canker resistance locus in order to be advanced. In other embodiments, a single genotype at only one locus may be sufficient for advancement.

By selecting only those individuals with the desired genotype for advancement in the breeding program, the frequency of desired alleles and desired phenotypes can be increased in future generations.

The introgression of one or more desired loci from a donor line into another is achieved via repeated backcrossing to a recurrent parent accompanied by selection to retain one or more stem canker resistance loci from the donor parent. Markers associated with stem canker resistance are assayed in progeny and those progeny with one or more stem canker resistance markers are selected for advancement. In another aspect, one or more markers can be assayed in the progeny to select for plants with the genotype of the agronomically elite parent. This invention anticipates that trait introgression activities will require more than one generation, wherein progeny are crossed to the recurrent (agronomically elite) parent or self-pollinated. Selections are made based on the presence of one or more stem canker resistance markers and can also be made based on the recurrent parent genotype, wherein screening is performed on a genetic marker and/or phenotype basis. In another embodiment, markers of this invention can be used in conjunction with other markers, ideally at least one on each chromosome of the soybean genome, to track the introgression of other desired traits as well as stem canker resistance into elite germplasm. In yet another embodiment, at least 200 SNP markers assorted across the 20 chromosomes of soybean will be useful in conjunction with the SNP molecular markers of the present invention to follow the introgression of other desired traits as well as stem canker resistance into elite germplasm. In a preferred embodiment. SNP markers distributed every 5 centimorgans across the 20 chromosomes of the soybean genetic linkage map, will be useful in conjunction with the SNP molecular markers of the present invention to follow the introgression of other desired traits as well as stem canker resistance into elite germplasm. In another embodiment, QTLs associated with stem canker resistance will be useful in conjunction with SNP molecular markers of the present invention to combine quantitative and qualitative stem canker resistance in the same plant. It is within the scope of this invention to utilize the methods and compositions for trait integration of stem canker resistance. It is contemplated that the present invention will be useful for developing commercial varieties with stem canker resistance and an agronomically elite phenotype.

For example, one skilled in the art can use one or more markers linked to the stem canker resistance locus, for example, those listed in Table 1, to select plants for stem canker resistance genotypes arising from the donor while selecting for the recipient genotypes in adjacent chromosome regions. In practice, this reduces the amount of linkage drag from the donor genome that maybe associated with undesirable agronomic properties. This backcrossing procedure is implemented at any stage in line development and occurs in conjunction with breeding for superior agronomic characteristics or one or more traits of interest, including transgenic and nontransgenic traits.

Alternatively, a forward breeding approach is employed wherein one or more stem canker resistance loci can be monitored for successful introgression following a cross with a susceptible parent with subsequent generations genotyped for one or more stem canker resistance loci and for one or more additional traits of interest, including transgenic and nontransgenic traits.

This invention can be used on populations other than those specifically described in this application without altering the methods described herein. Although different parents may have different genotypes at different markers, the method of using this invention is fundamentally identical. Parents are first phenotyped for stem canker resistance, genotyped at each marker, and then those genotypes are used to infer resistant or susceptible phenotypes in progeny derived from those parents or in any other population where the genotypes are associated with the same phenotypes.

Having illustrated and described the principles of the present invention, it should be apparent to persons skilled in the art that the invention can be modified in arrangement and detail without departing from such principles.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

We claim:

1. A method for creating a population of soybean plants with enhanced stem canker resistance comprising:
a. providing a first population of soybean plants;
b. detecting the presence of a genetic marker that is genetically linked to a stem canker resistance locus on linkage group B2 by 20cM or less in the first population;
c. selecting one or more soybean plants containing said marker from the first population of soybean plants; and
d. producing a population of offspring from at least one of said selected soybean plants.

2. The method of claim 1 wherein the genetic marker detected is genetically linked to the stem canker resistance locus by less than 15cM.

3. The method of claim 1 wherein the genetic marker detected is genetically linked to the stem canker resistance locus by less than 10cM.

4. The method of claim 1 wherein the genetic marker detected is located within a chromosome interval comprising and flanked by Glyma14g00220 and Glyma14g05460.

5. The method of claim 1 wherein the genetic marker detected is located within a chromosome interval comprising and flanked by Glyma14g02010 and Glyma14g03380.

6. The method of claim 1 wherein the genetic marker detected is located within a chromosome interval comprising and flanked by SEQ ID NO. 1 and SEQ ID NO. 77.

7. The method of claim 1 wherein the genetic marker detected is located within a chromosome interval comprising and flanked by SEQ ID NO. 6 and SEQ ID NO. 71.

8. The method of claim 1 wherein the genetic marker detected is located within a chromosome interval comprising and flanked by SEQ ID NO. 9 and SEQ ID NO. 64.

9. The method of claim 1 wherein the genetic marker detected is located within a chromosome interval comprising and flanked by SEQ ID NO. 24 and SEQ ID NO. 64.

10. The method of claim 1 wherein the genetic marker detected is selected from at least one of the group consisting of SEQ ID NOs. 1-77.

11. The method of claim 1 wherein a portion of the stem canker is caused by Diaporthe phaseolorum.

12. A method of creating a population of soybean plants comprising at least one allele associated with enhanced stem canker resistance comprising at least one sequence selected from the group consisting of SEQ ID NO: 1 to 77, the method comprising the steps of:
a. genotyping a first population of soybean plants, said population containing at least one allele associated with enhanced stem canker resistance, the at least one allele associated with enhanced stem canker resistance comprising at least one sequence selected from the group consisting of SEQ ID NO: 1 to 77;
b. selecting from said first population one or more identified soybean plants containing said at least one allele associated with enhanced stem canker resistance comprising at least one sequence selected from the group consisting of SEQ ID NO: 1 to 77; and
c. producing from said selected soybean plants a second population, thereby creating a population of soybean plants comprising at least one allele associated with enhanced stem canker resistance comprising at least one sequence selected from the group consisting of SEQ ID NO: 1 to 77.
